(19) United States
(12) Patent Application Publication
(10) Pub. No.: US 2022/0015372 A1
(43) Pub. Date: Jan. 20, 2022

(54) BIOLOGICALS AND THEIR USE IN PLANTS

(71) Applicant: PIONEER HI-BRED INTERNATIONAL, INC., JOHINSTON, IA (US)

(72) Inventors: Monique Coy, Johinston, IA (US); Steven D. Gruver, Pacifica, CA (US); Heather Kozy, Walnut Creek, CA (US); Lu Liu, Palo Alto, CA (US); Young-Jun Park, Fremont, CA (US); Barbara Rosen, Mountain View, CA (US); Ute Schellenberger, Palo Alto, CA (US); Jun-Zhi Wer, Johnston, IA (US); Weiping Xie, East Palo Alto, CA (US)

(73) Assignee: PIONEER HI-BRED INTERNATIONAL, INC., JOHINSTON, IA (US)

(21) Appl. No.: 17/312,763

(22) PCT Filed: Dec. 5, 2019
(86) PCT No.: PCT/US2019/064579

§ 371 Date: Jun. 10, 2021

Related U.S. Application Data
(60) Provisional application No. 62/779,642, filed on Dec. 14, 2018.

(51) Int. Cl. A01N 63/22; A01N 63/27; C12N 1/20; C07K 11/02; C12N 15/113

(52) U.S. Cl. CPC A01N 63/22; A01N 63/27; C12R 2001/36; C07K 11/02; C12N 15/113; C12N 1/205

(57) ABSTRACT

Biological strains, compositions, and methods of using the strains and compositions for reducing overall insect damage.

### CROSS-REFERENCE TO RELATED APPLICATIONS

This Application claims the benefit of U.S. Provisional Application No. 62/779,642 filed on Dec. 14, 2018, which is incorporated herein by reference in its entirety.

### FIELD

Biological strains, compositions, and methods of using the strains and compositions for reducing overall insect damage.

### REFERENCE TO SEQUENCE LISTING SUBMITTED ELECTRONICALLY

The official copy of the sequence listing is submitted electronically via EFS-Web as an ASCII formatted sequence listing with a file named 6593_SeqList.txt created on Aug. 21, 2018 and having a size of 1,395 kilobytes and is filed concurrently with the specification. The sequence listing contained in this ASCII formatted document is part of the specification and is herein incorporated by reference in its entirety.

### BACKGROUND

Certain species of microorganisms of the genus Bacillus are known to possess pesticidal activity against a range of insect pests including Lepidoptera, Diptera, Coleoptera, Hemiptera and others. Bacillus thuringiensis (Bt) and Bacillus popilliae are among the most successful biocontrol agents discovered to date. Insect pathogenicity has also been attributed to strains of B. larvae, B. lentimorbus, B. sphaericus and B. cereus. Microbial insecticides, particularly those obtained from Bacillus strains, have played an important role in agriculture as alternatives to chemical pest control.

Crop plants have been developed with enhanced insect resistance by genetically engineering crop plants to produce pesticidal proteins from Bacillus. For example, corn and cotton plants have been genetically engineered to produce pesticidal proteins isolated from strains of Bt. These genetically modified crops are now widely used in agriculture and have provided the farmer with an environmentally friendly alternative to traditional insect-control methods. While they have proven to be very successful commercially, these genetically modified, insect-resistant crop plants provide resistance to only a narrow range of the economically important insect pests. In some cases, insects can develop resistance to different insecticidal compounds, which raises the need to identify alternative biological control agents for pest control.

There has been a long felt need for environmentally friendly compositions and methods for controlling or eradicating insect pests of agricultural significance, i.e., methods that are selective, environmentally inert, non-persistent, and biodegradable, and that fit well into insect pest management schemes.

### SUMMARY

Some embodiments relate to a composition comprising a Pseudomonas chlororaphis, a Burkholderia rinojensis, or a Chromobacterium haemolyticum, wherein the Pseudomonas chlororaphis, Burkholderia rinojensis, or Chromobacterium haemolyticum has insecticidal activity. In some embodiments, the methods and compositions relate to a insecticidal bacterial strain comprising a DepA gene, a DepB gene, a DepC gene, a DepF gene, a DepG gene, DepH gene, a DepE gene, or a DepD gene. In some embodiments, the DepA gene comprises an amino acid sequence having at least 90% sequence identity to any one of SEQ ID NOs: 109, 121, 133, 145, 158, 171, 184, or 197. In some embodiments, the DepB gene comprises an amino acid sequence having at least 90% sequence identity to any one of SEQ ID NOs: 110, 122, 134, 146, 159, 172, 185, or 198. In some embodiments, the DepC gene comprises an amino acid sequence having at least 90% sequence identity to any one of SEQ ID NOs: 111, 123, 135, 147, 160, 173, 186, or 199. In some embodiments, the DepD gene comprises an amino acid sequence having at least 90% sequence identity to any one of SEQ ID NOs: 112, 124, 136, 148, 161, 174, 187, or 200. In some embodiments, the DepE gene comprises an amino acid sequence having at least 90% sequence identity to any one of SEQ ID NOs: 113, 125, 137, 149, 162, 175, 188, or 201. In some embodiments, the DepF gene comprises an amino acid sequence having at least 90% sequence identity to any one of SEQ ID NOs: 114, 126, 138, 150, 163, 176, 189, or 202. In some embodiments, the DepG gene comprises an amino acid sequence having at least 90% sequence identity to any one of SEQ ID NOs: 115, 127, 139, 151, 164, 177, 190, or 203. In some embodiments, the DepH gene comprises an amino acid sequence having at least 90% sequence identity to any one of SEQ ID NOs: 116, 128, 140, 152, 165, 178, 191, or 204. In some embodiments, the DepI gene comprises an amino acid sequence having at least 90% sequence identity to any one of SEQ ID NOs: 117, 129, 141, 153, 166, 179, 192, or 205. In some embodiments, the DepJ gene comprises an amino acid sequence having at least 90% sequence identity to any one of SEQ ID NOs: 118, 130, 142, 154, 167, 180, 193, or 206. In some embodiments, the DepK gene comprises an amino acid sequence having at least 90% sequence identity to any one of SEQ ID NOs: 143, 155, 168, 181, or 194. In some embodiments, the DepL gene comprises an amino acid sequence having at least 90% sequence identity to any one of SEQ ID NOs: 119, 131, 156, 169, 182, 195, or 207. In some embodiments, the DepM gene comprises an amino acid sequence having at least 90% sequence identity to any one of SEQ ID NOs: 120, 132, 144, 157, 170, 183, 196, or 208. In some embodiments, the methods and compositions relate to bacterial strains comprising a 16S RNA sequence having at least 95% identity to any one of SEQ ID NOs: 1-8.

In one embodiment, the disclosure relates to a composition comprising a Pseudomonas chlororaphis strain SS532D1 (NRRL Deposit No. B-67638), wherein the Pseudomonas chlororaphis strain SS532D1 has insecticidal activity.

In one embodiment, the disclosure relates to a composition comprising a Pseudomonas chlororaphis strain SSP459B9-3 (NRRL Deposit No. B-67639), wherein the Pseudomonas chlororaphis strain SS532D1 has insecticidal activity.

In one embodiment, the disclosure relates to a composition comprising a Burkholderia rinojensis strain JH59178-1 (NRRL Deposit No. B-67640), wherein the Burkholderia rinojensis strain JH59178-1 has insecticidal activity.

In one embodiment, the disclosure relates to a composition comprising a Chromobacterium haemolyticum strain JH91791-1 (NRRL Deposit No. B-67642), wherein the Chromobacterium haemolyticum strain JH91791-1 has insecticidal activity.

In one embodiment, the disclosure relates to a composition comprising a Chromobacterium haemolyticum strain PMCJ4191H4-1 NRRL Deposit No. B-67644), wherein the Chromobacterium haemolyticum strain PMCJ4191H4-1 has insecticidal activity.

In one embodiment, the disclosure relates to a composition comprising a Chromobacterium haemolyticum strain JH97285 -1 (NRRL Deposit No. B-67641), wherein the Chromobacterium haemolyticum strain JH97285 -1 has insecticidal activity.

In one embodiment, the disclosure relates to a composition comprising a Chromobacterium haemolyticum strain PMC3591F10-1 (NRRL Deposit No. B-67643), wherein the Chromobacterium haemolyticum strain PMC3591F10 -1 has insecticidal activity.

In yet another embodiment, the disclosure relates methods and compositions comprising a bacterial strain disclosed herein, or a progeny, mutant, or variant thereof, a fermentate produced from a strain disclosed herein progeny, mutant, or variant thereof, and/or a depsipeptide in an effective amount to achieve an effect of inhibit growth of a plant pathogen, pest or insect. In some embodiments, the depsipeptide has the structure of FR901375, FK228, or a derivative or variation thereof. In another embodiment, the composition further comprises a biocontrol agent selected from the group consisting of bacteria, fungi, yeast, protozoans, viruses, entomopathogenic nematodes, botanical extracts, proteins, secondary metabolites, and inoculants.

In another embodiment, the compositions and methods disclosed herein further comprise one or more agrochemically active compounds selected from the group consisting of an insecticide, a fungicide, a bactericide, and a nematicide. In still another embodiment, the composition further comprises a compound selected from the group consisting of a safener, a lipo-chitooligosaccharide, an isoflavone, and a ryanodine receptor modulator.

In another embodiment, the compositions and methods comprise at least one at least one seed, plant, or plant part. In one embodiment, the seed, plant, or plant part is genetically modified.

In one embodiment, the compositions and methods inhibit the growth of one or more plant pathogens, pests, or insects including but not limited to bacteria, a fungus, a virus, protozoa, nematode, or an arthropod. In one embodiment, the compositions and methods inhibit the growth of an insect, including but not limited to a Coleopteran, Hemipteran, or Lepidopteran insect. In still another embodiment, the composition inhibits the growth of Diabrotica virgifera virgifera, Ostrinia nubilalis, Spodoptera frugiperda, Pseudoplusia includens, Anticarsia gemmatalis, Plutella xylostella, and/or Aphis fabae.

In another embodiment, the compositions and methods comprise an effective amount to provide pesticidal activity to bacteria, plants, plant cells, tissues and seeds. In another embodiment, the composition is an effective amount to provide pesticidal activity to Coleopteran or Lepidopteran insects. In still another embodiment, the composition is an effective amount to provide pesticidal activity to Diabrotica virgifera virgifera, Ostrinia nubilalis, Spodoptera frugiperda, Pseudoplusia includens, Anticarsia gemmatalis, Plutella xylostella, and/or Aphis fabae.

In another embodiment, the compositions and methods comprise in an effective amount to improve plant performance including but not limited to increased root formation, increased root mass, increased root function, increased shoot height, increased shoot function, increased flower bud presence, increased flower bud formation, increased seed germination, increased yield, increased total plant wet weight, and increased total plant dry weight.

In another embodiment, the disclosure relates to a method comprising applying a composition comprising a bacterial strain disclosed herein, or a progeny, mutant, or variant thereof, a fermentate produced from a strain disclosed herein, or a progeny, mutant, or variant thereof, and/or a depsipeptide to a seed, a plant, plant part or soil in an effective amount to achieve an effect selected from the group consisting of: inhibit a plant pathogen, pest, or insect or to prevent damage to a plant by a pathogen, pest, or insect; improve plant performance; improve plant yield; improve plant vigor; increase phosphate availability; increase production of a plant hormone; increase root formation; increase shoot height in a plant, increase leaf length of a plant; increase flower bud formation of a plant; increase total plant fresh weight; increase total plant dry weight; and increase seed germination.

In another embodiment, the method further comprises applying a biocontrol agent, wherein the biocontrol agent is selected from the group consisting of bacteria, fungi, yeast, protozoans, viruses, entomopathogenic nematodes, botanical extracts, proteins, secondary metabolites, and inoculants.

In yet another embodiment, the method further comprises applying an agrochemically active compound selected from the group consisting of an insecticide, a fungicide, a bactericide, and a nematicide. In still another embodiment, the method further comprises applying a compound selected from the group consisting of a safener, a lipo-chitooligosaccharide, an isoflavone, and a ryanodine receptor modulator.

In another embodiment, the method comprises applying the composition in an effective amount to inhibit growth of a plant pathogen, including but not limited to bacteria, a fungus, a nematode, an insect, a virus, and protozoa.

### DESCRIPTION OF FIGURES

FIG. 1 shows the depsipeptide structures of FR-901375(1) and FK228 and associated biosynthetic pathways.

FIG. 2 shows the effect of growth conditions on insecticidal potency in various fermented broths of strain SS532D1 on FAW and WCRW.

FIG. 3 shows cultures of multiple bacterial strains and their insecticidal activities. Scores are represented as: 2.6-3.0, killing; 1.7-2.5, severe stunting; 1.0-1.6, stunting; 0-0.9 inactive.

FIG. 4 shows a homology table of A) DepA genes and B) DepB genes from the various strains.

FIG. 5 shows a homology table of A) DepC genes and B) DepF genes from the various strains.

FIG. 6 shows a homology table of A) DepG genes and B) DepH genes from the various strains.

FIG. 7 shows a homology table of A) DepE genes and B) DepD genes from the various strains.

### DESCRIPTION OF THE SEQUENCES

The disclosure can be more fully understood from the following detailed description and the accompanying drawings and Sequence Listing that form a part of this application.

The sequence descriptions summarize the Sequence Listing attached hereto, which is hereby incorporated by reference. The Sequence Listing contains one letter codes for nucleotide sequence characters and the single and three letter codes for amino acids as defined in the IUPAC-IUB standards described in Nucleic Acids Research 13:3021-3030 (1985) and in the Biochemical Journal 219(2):345-373 (1984).

TABLE 1
Sequence Listing Description

| Strain_Gene | SEQ ID NO: |
|---|---|
| SSP532D1b_16S | 1 |
| SSP459B9-3_16S | 2 |
| JH59178-1_16S | 3 |
| JH97285-1_16S | 4 |
| JH91791-1_16S | 5 |
| PMC3591F10-1_16S | 6 |
| PMCJ4191H4-1_16S | 7 |
| PMCJ4232B7-1_16S | 8 |
| SSP532D1b_DepA | 9 |
| SSP532D1b_DepB | 10 |
| SSP532D1b_DepC | 11 |
| SSP532D1b_DepD | 12 |
| SSP532D1b_DepE | 13 |
| SSP532D1b_DepF | 14 |
| SSP532D1b_DepG | 15 |
| SSP532D1b_DepH | 16 |
| SSP532D1b_DepI | 17 |
| SSP532D1b_DepJ | 18 |
| SSP532D1b_DepL | 19 |
| SSP532D1b_DepM | 20 |
| SSP459B9-3_DepA | 21 |
| SSP459B9-3_DepB | 22 |
| SSP459B9-3_DepC | 23 |
| SSP459B9-3_DepD | 24 |
| SSP459B9-3_DepE | 25 |
| SSP459B9-3_DepF | 26 |
| SSP459B9-3_DepG | 27 |
| SSP459B9-3_DepH | 28 |
| SSP459B9-3_DepI | 29 |
| SSP459B9-3_DepJ | 30 |
| SSP459B9-3_DepL | 31 |
| SSP459B9-3_DepM | 32 |
| JH59178-1_DepA | 33 |
| JH59178-1_DepB | 34 |
| JH59178-1_DepC | 35 |
| JH59178-1_DepD | 36 |
| JH59178-1_DepE | 37 |
| JH59178-1_DepF | 38 |
| JH59178-1_DepG | 39 |
| JH59178-1_DepH | 40 |
| JH59178-1_DepI | 41 |
| JH59178-1_DepJ | 42 |
| JH59178-1_DepK | 43 |
| JH59178-1_DepM | 44 |
| JH97285-1_DepA | 45 |
| JH97285-1_DepB | 46 |
| JH97285-1_DepC | 47 |
| JH97285-1_DepD | 48 |
| JH97285-1_DepE | 49 |
| JH97285-1_DepF | 50 |
| JH97285-1_DepG | 51 |
| JH97285-1_DepH | 52 |
| JH97285-1_DepI | 53 |
| JH97285-1_DepJ | 54 |
| JH97285-1_DepK | 55 |
| JH97285-1_DepL | 56 |
| JH97285-1_DepM | 57 |
| JH91791-1_DepA | 58 |
| JH91791-1_DepB | 59 |
| JH91791-1_DepC | 60 |
| JH97285-1_DepD | 61 |
| JH91791-1_DepE | 62 |
| JH91791-1_DepF | 63 |
| JH91791-1_DepG | 64 |
| JH91791-1_DepH | 65 |
| JH91791-1_DepI | 66 |
| JH91791-1_DepJ | 67 |
| JH91791-1_DepK | 68 |
| JH91791-1_DepL | 69 |
| JH91791-1_DepM | 70 |
| PMC3591F10-1_DepA | 71 |
| PMC3591F10-1_DepB | 72 |
| PMC3591F10-1_DepC | 73 |
| PMC3591F10-1_DepD | 74 |
| PMC3591F10-1_DepE | 75 |
| PMC3591F10-1_DepF | 76 |
| PMC3591F10-1_DepG | 77 |
| PMC3591F10-1_DepH | 78 |
| PMC3591F10-1_DepI | 79 |
| PMC3591F10-1_DepJ | 80 |
| PMC3591F10-1_DepK | 81 |
| PMC3591F10-1_DepL | 82 |
| PMC3591F10-1_DepM | 83 |
| PMCJ4191H4-1_DepA | 84 |
| PMCJ4191H4-1_DepB | 85 |
| PMCJ4191H4-1_DepC | 86 |
| PMCJ4191H4-1_DepD | 87 |
| PMCJ4191H4-1_DepE | 88 |
| PMCJ4191H4-1_DepF | 89 |
| PMCJ4191H4-1_DepG | 90 |
| PMCJ4191H4-1_DepH | 91 |
| PMCJ4191H4-1_DepI | 92 |
| PMCJ4191H4-1_DepJ | 93 |
| PMCJ4191H4-1_DepK | 94 |
| PMCJ4191H4-1_DepL | 95 |
| PMCJ4191H4-1_DepM | 96 |
| PMCJ4232B7-1_DepA | 97 |
| PMCJ4232B7-1_DepB | 98 |
| PMCJ4232B7-1_DepC | 99 |
| PMCJ4232B7-1_DepD | 100 |
| PMCJ4232B7-1_DepE | 101 |
| PMCJ4232B7-1_DepF | 102 |
| PMCJ4232B7-1_DepG | 103 |
| PMCJ4232B7-1_DepH | 104 |
| PMCJ4232B7-1_DepI | 105 |
| PMCJ4232B7-1_DepJ | 106 |
| PMCJ4232B7-1_DepL | 107 |
| PMCJ4232B7-1_DepM | 108 |

### DETAILED DESCRIPTION

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof, and so forth. All technical and scientific terms used herein have the same meaning as commonly understood to one of ordinary skill in the art to which this disclosure belongs unless clearly indicated otherwise.

As used herein, “administer” refers to the action of introducing a strain and/or a composition to an environment for pathogen, pest, or insect inhibition or to improve plant performance.

As used herein, the term “agrochemically active compounds” refers to any substance that is or may be customarily used for treating plants including, but not limited to, fungicides, bactericides, insecticides, acaricides, nematicides, molluscicides, safeners, plant growth regulators, and plant nutrients, as well as, microorganisms.

As used herein, a composition may be a liquid, a heterogeneous mixture, a homogeneous mixture, a powder, a solution, a dispersion or any combination thereof.

As used herein, a “depsipeptide” refers to certain cyclic peptide(s) obtained from the fermentation broth of a Pseudomonas chlororaphis strain provided herein. In some embodiments, the depsipeptide has certain biological activity including histone deacetylase inhibitors (HDACi). In some embodiments, the depsipeptide comprises a compound having the structure of FR901375 as shown below.

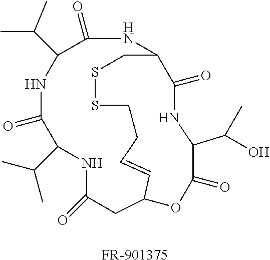

FR091375 was initially reported in patent application JPH03141296A published in 1991, which subsequently became granted patent JP2833181B2 in 1998, wherein the contents of both are incorporated by reference in their entirety. Although certain anti-cancer activities were reported in these Japanese publications, no HDACi activity of FR901375 was reported until Narita et al., 1996, “Total Synthesis of the Depesipeptide FR901375 and Preliminary Evaluation of its Biological Activity” Eur. J. Chem. 2018, 5667-5677. However, Narita et al. 1996 only shows certain in vitro HDACi activity and there has been no in vivo activity of FR901375 that has been reported today, even twenty-seven years after its initial discovery in 1991.

In some embodiments, the depsipeptide comprises a compound having the structure of FK228 (also known as FR901228 or romidepsin) as shown below.

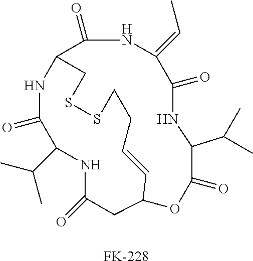

FK228 (also known as FR901228 or romidepsin) was initially isolated from Chroinobacterium violaceum and reported in U.S. Pat. No. 4.977.138 titled “FR901228 Substance and Preparation Thereof,” wherein the content of which is incorporated by reference in its entirety. U.S. Pat. No. 4,977,138 only disclosed certain antimicrobial and antitumor activities of FR901228/FK228, and its HDACi activity was reported in Furumai et al., 2002, “FK228 (Depsipeptide) as a Natural Prodrug that Inhibits Class I Histone Deacetylases” Cancer Res. (2002) 62: 4916-4921. Furumai et al. 2002 also discloses chemical modification of FK228 to generate derivatives for example redFK and dimethyl FK228 as shown in Scheme 1 below.

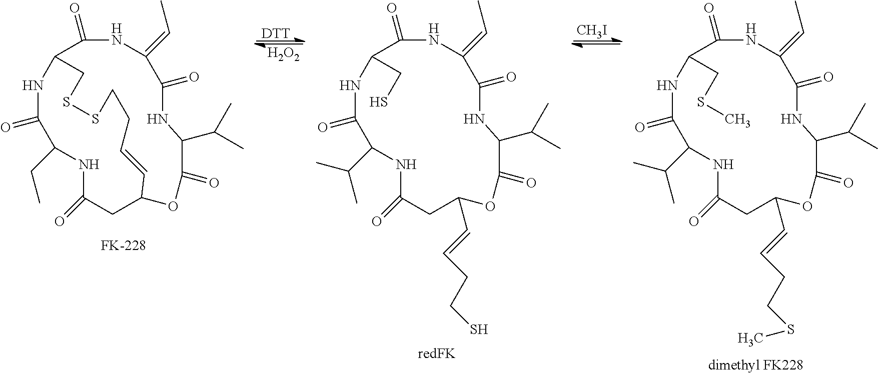 [SMILES: C/C=C1\NC(=O)C2CSSCC/C=C/C(CC(=O)NC(CC)C(=O)N2)OC(=O)C(C(C)C)NC1=O ; "DTT" ; "H2O2" ; C/C=C1\NC(=O)C(CS)NC(=O)C(C(C)C)NC(=O)CC(/C=C/CCS)OC(=O)C(C(C)C)NC1=O ; "CH3I" ; C/C=C1\NC(=O)C(CSC)NC(=O)C(C(C)C)NC(=O)CC(/C=C/CCSC)OC(=O)C(C(C)C)NC1=O]

As used herein, “effective amount” refers to a quantity of a bacterial strain disclosed herein, or a progeny, mutant, or variant thereof, a fermentate produced from a strain disclosed herein progeny, mutant, or variant thereof, and/or a depsipeptide sufficient to inhibit growth of a pathogenic microorganism or to impede the rate of growth of the pathogenic microorganism. In another embodiment, the term “effective amount” refers to a quantity of a bacterial strain disclosed herein, or a progeny, mutant, or variant thereof, a fermentate produced from a strain disclosed herein progeny, mutant, or variant thereof, and/or a depsipeptide sufficient to improve plant performance. In another embodiment, the term “effective amount” refers to a quantity of a bacterial strain disclosed herein, or a progeny, mutant, or variant thereof, a fermentate produced from a strain disclosed herein progeny, mutant, or variant thereof, and/or a depsipeptide sufficient to control, kill, inhibit, and reduce the number, emergence, or growth of a pathogen, pest, or insect. In another embodiment, the term “effective amount” refers to a quantity of a bacterial strain disclosed herein, or a progeny, mutant, or variant thereof, a fermentate produced from a strain disclosed herein progeny, mutant, or variant thereof, and/or a depsipeptide sufficient to prevent damage from a pathogen, pest, or insect. One skilled in the art will recognized that an effective amount of a bacterial strain disclosed herein, or a progeny, mutant, or variant thereof, a fermentate produced from a strain disclosed herein progeny, mutant, or variant thereof, and/or a depsipeptide may not reduce the numbers of pathogens, pests or insects, but is effective in decreasing damage to plants and/or plant parts as a result of a pathogen, pest or insect. For example, a pesticidally effective amount may reduce pathogen, pest or insect emergence, or damage to seeds, roots, shoots, or foliage of plants that are treated compared to those that are untreated.

As used herein, “fermentate broth,” “fermentate,” or “fermented broth” refers to a media used to grow or ferment a bacterial strain disclosed herein. The bacterial strain may be removed from a media by filtration, sterilization, or other means. The leftover broth contains metabolites produced by a bacterial strain disclosed herein, which is collectively referred to as a “fermentate broth,” “fermentate,” or “fermented broth.”

As used herein, the term “inhibit” refers to destroy, prevent, reduce, resist, control, decrease, slow or otherwise interfere with the growth or survival of a pathogen, pest, or insect when compared to the growth or survival of the pathogen, pest, or insect in an untreated control. Any of the terms of inhibit, destroy, prevent, control, decrease, slow, interfere, resist, or reduce may be used interchangeably. In one embodiment, to “inhibit” is to destroy, prevent, control, reduce, resist, decrease, slow or otherwise interfere with the growth, emergence, or survival of a pathogen, pest, or insect by at least about 3% to at least about 100%, or any value in between for example at least about 10%, 15%, 20%, 25%, 30%, 35%, 40%, 45%, 50%, 55%, 60%, 65%, 70%, 75%, 80%, 85%, 90%, 95%, 99%, or 100% when compared to the growth or survival of the pathogen, pest, or insect in an untreated control. The amount of inhibition can be measured as described herein or by other methods known in the art. As used herein, “protects a plant from a pathogen, pest, or insect pest” is intended to mean the limiting or eliminating of the pathogen, pest, or insect related damage to a plant and/or plant part by, for example, inhibiting the ability of the pathogen, pest, or insect to grow, emerge, feed, and/or reproduce or by killing the pathogen, pest, or insect. As used herein, pesticidal and/or insecticidal activity refers to an activity of compound, composition, and or method that protects a plant and/or plant part from a pathogen, pest, or insect.

In some embodiments, inhibition a pathogen, pest, or insect lasts for or provides protection for greater than a day, two days, three days, four days, five days, six days, a week, two weeks, three weeks, a month or more after of a bacterial strain disclosed herein, or a progeny, mutant, or variant thereof, a fermentate produced from a strain disclosed herein progeny, mutant, or variant thereof, and/or a depsipeptide disclosed herein is applied to subject material. In another embodiment, inhibition a pathogen, pest or insect lasts from one to seven days, from seven to 14 days, from 14 to 21 days, or from 21 to 30 days or more. In another embodiment, the inhibition of the growth of a pathogen, pest, or insect lasts for or provides protection for greater than the time from application to adult emergence of the pathogen, pest, or insect.

As used herein, the term “genetically modified” is intended to mean any species containing a genetic trait, loci, or sequence that was not found in the species or strain prior to manipulation. A genetically modified plant may be transgenic, cis-genic, genome edited, or bred to contain a new genetic trait, loci, or sequence. A genetically modified plant or bacteria may be prepared by means known to those skilled in the art, such as transformation by bombardment, by a gene editing technique such as Cas/CRISPR or TALENS, or by breeding techniques. As used herein, a “trait” is a new or modified locus or sequence of a genetically modified plant or bacteria, including but not limited to a transgenic plant or bacteria. In some embodiments, a bacterial strain disclosed herein, or a progeny, mutant, or variant thereof, may be edited. In some embodiments any bacterial strain may be modified or edited to comprise any one of a DepA gene, a DepB gene, a DepC gene, a DepF gene, a DepG gene, DepH gene, a DepE gene, or a DepD gene. In some embodiments, the methods and compositions relate to a insecticidal bacterial strain selected from a group consisting of a DepA gene, a DepB gene, a DepC gene, a DepF gene, a DepG gene, DepH gene, a DepE gene, and a DepD gene

As used herein, the term “environment of a plant or plant part” is intended to mean the area surrounding the plant or plant part, including but not limited to the soil, the air, or in-furrow. The environment of a plant or plant part may be in close proximity, touching, adjacent to, or in the same field as the plant or plant part. The compositions described herein may be applied to the environment of the plant or plant part as a seed treatment, as a foliar application, as a granular application, as a soil application, or as an encapsulated application. As used herein, “in-furrow” is intended to mean within or near the area where a seed is planted. The compositions disclosed herein may be applied in-furrow concurrently or simultaneously with a seed. In another embodiment, the compositions disclosed herein may be applied sequentially, either before or after a seed is planted.

As used herein, the term “different mode of action” is used to refer to a pesticidal composition inhibiting a pathogen, pest, or insect through a pathway or receptor that is different from another pesticidal composition. As used herein, the term “different mode of action” includes the pesticidal effects of one or more pesticidal compositions to different binding sites (i.e., different toxin receptors and/or different sites on the same toxin receptor) in the gut membranes of insects or through the RNA interference pathway to different target genes.

As used herein, the term “pathogen, pest, or insect” includes but is not limited to pathogenic fungi, bacteria, mites, ticks, pathogenic microorganisms, and nematodes, as well as insect from the orders Coleoptera, Lepidoptera, Mallophaga, Homoptera, Hemiptera, Orthroptera, Thysanoptera, Dermaptera, Isoptera, Anoplura, Siphonatpera, Trichoptera, and others, including but not limited to Diabrotica virgifera virgifera, Diabrotica undecimpunctata howardi, and Diabrotica barberi.

Larvae of the order Lepidoptera include, but are not limited to, armyworms, cutworms, loopers and heliothines in the family Noctuidae Spodoptera frugiperda J E Smith (fall armyworm); S. exigua Hübner (beet armyworm); S. litura Fabricius (tobacco cutworm, cluster caterpillar); Mamestra configurata Walker (bertha armyworm); M. brassicae Linnaeus (cabbage moth); Agrotis ipsilon Hufnagel (black cutworm); A. orthogonia Morrison (western cutworm); A. subterranea Fabricius (granulate cutworm); Alabama argillacea Hübner (cotton leaf worm); Trichoplusia ni Hübner (cabbage looper); Pseudoplusia includens Walker (soybean looper); Anticarsia gemmatalis Hübner (velvetbean caterpillar); Hypena scabra Fabricius (green cloverworm); Heliothis virescens Fabricius (tobacco budworm); Pseudaletia unipuncta Haworth (armyworm); Athetis mindara Barnes and Mcdunnough (rough skinned cutworm); Euxoa messoria Harris (darksided cutworm); Earias insulana Boisduval (spiny bollworm); E. vittella Fabricius (spotted bollworm); Helicoverpa armigera Hübner (American bollworm); H. zea Boddie (corn earworm or cotton bollworm); Melanchra picta Harris (zebra caterpillar); Egira (Xylomyges) curialis Grote (citrus cutworm); borers, casebearers, webworms, coneworms, and skeletonizers from the family Pyralidae Ostrinia nubilalis Hübner (European corn borer); Amyelois transitella Walker (naval orangeworm); Anagasta kuehniella Zeller (Mediterranean flour moth); Cadra cautella Walker (almond moth); Chilo suppressalis Walker (rice stem borer); C. partellus, (sorghum borer); Corcyra cephalonica Stainton (rice moth); Crambus caliginosellus Clemens (corn root webworm); C. teterrellus Zincken (bluegrass webworm); Cnaphalocrocis medinalis Guenëe (rice leaf roller); Desmia funeralis Hübner (grape leaffolder); Diaphania hyalinata Linnaeus (melon worm); D. nitidalis Stoll (pickleworm); Diatraea grandiosella Dyar (southwestern corn borer), D. saccharalis Fabricius (surgarcane borer); Eoreuma loftini Dyar (Mexican rice borer); Ephestia elutella Hübner (tobacco (cacao) moth); Galleria mellonella Linnaeus (greater wax moth); Herpetogramma licarsisalis Walker (sod webworm); Homoeosoma electellum Hulst (sunflower moth); Elasmopalpus lignosellus Zeller (lesser cornstalk borer); Achroia grisella Fabricius (lesser wax moth); Loxostege sticticalis Linnaeus (beet webworm); Orthaga thyrisalis Walker (tea tree web moth); Maruca testulalis Geyer (bean pod borer); Plodia interpunctella Hübner (Indian meal moth); Scirpophaga incertulas Walker (yellow stem borer); Udea rubigalis Guenée (celery leaftier); and leafrollers, budworms, seed worms and fruit worms in the family Tortricidae Acleris gloverana Walsingham (Western blackheaded budworm); A. variana Fernald (Eastern blackheaded budworm); Archips argyrospila Walker (fruit tree leaf roller); A. rosana Linnaeus (European leaf roller); and other Archips species, Adoxophyes orana Fischer von Rösslerstamm (summer fruit tortrix moth); Cochylis hospes Walsingham (banded sunflower moth); Cydia latiferreana Walsingham (filbertworm); C. pomonella Linnaeus (coding moth); Platynota flavedana Clemens (variegated leafroller); P. stultana Walsingham (omnivorous leafroller); Lobesia botrana Denis & Schiffermüller (European grape vine moth); Spilonota ocellana Denis & Schiffermüller (eyespotted bud moth); Endopiza viteana Clemens (grape berry moth); Eupoecilia ambiguella Hübner (vine moth); Bonagota salubricola Meyrick (Brazilian apple leafroller); Grapholita molesta Busck (oriental fruit moth); Suleima helianthana Riley (sunflower bud moth); Argyrotaenia spp.; Choristoneura spp.

Selected other agronomic pests in the order Lepidoptera include, but are not limited to, Alsophila pometaria Harris (fall cankerworm); Anarsia lineatella Zeller (peach twig borer); Anisota senatoria J. E. Smith (orange striped oakworm); Antheraea pernyi Guérin-Méneville (Chinese Oak Tussah Moth); Bombyx mori Linnaeus (Silkworm); Bucculatrix thurberiella Busck (cotton leaf perforator); Colias eurytheme Boisduval (alfalfa caterpillar); Datana integerrima Grote & Robinson (walnut caterpillar); Dendrolimus sibiricus Tschetwerikov (Siberian silk moth), Ennomos subsignaria Hübner (elm spanworm); Erannis tiliaria Harris (linden looper); Euproctis chrysorrhoea Linnaeus (browntail moth); Harrisina americana Guérin-Méneville (grapeleaf skeletonizer); Hemileuca oliviae Cockrell (range caterpillar); Hyphantria cunea Drury (fall webworm); Keiferia lycopersicella Walsingham (tomato pinworm); Lambdina fiscellaria fiscellaria Hulst (Eastern hemlock looper); L. fiscellaria lugubrosa Hulst (Western hemlock looper); Leucoma salicis Linnaeus (satin moth); Lymantria dispar Linnaeus (gypsy moth); Manduca quinquemaculata Haworth (five spotted hawk moth, tomato hornworm); M. sexta Haworth (tomato hornworm, tobacco hornworm); Operophtera brumata Linnaeus (winter moth); Paleacrita vernata Peck (spring cankerworm); Papilio cresphontes Cramer (giant swallowtail orange dog); Phryganidia californica Packard (California oakworm); Phyllocnistis citrella Stainton (citrus leafminer); Phyllonorycter blancardella Fabricius (spotted tentiform leafminer); Pieris brassicae Linnaeus (large white butterfly); P. rapae Linnaeus (small white butterfly); P. napi Linnaeus (green veined white butterfly); Platyptilia carduidactyla Riley (artichoke plume moth); Plutella xylostella Linnaeus (diamondback moth); Pectinophora gossypiella Saunders (pink bollworm); Pontia protodice Boisduval and Leconte (Southern cabbageworm); Sabulodes aegrotata Guenée (omnivorous looper); Schizura concinna J. E. Smith (red humped caterpillar); Sitotroga cerealella Olivier (Angoumois grain moth); Thaumetopoea pityocampa Schiffermuller (pine processionary caterpillar); Tineola bisselliella Hummel (webbing clothesmoth); Tuta absoluta Meyrick (tomato leafminer); Yponomeuta padella Linnaeus (ermine moth); Heliothis subflexa Guenée; Malacosoma spp. and Orgyia spp.

Of interest are larvae and adults of the order Coleoptera including weevils from the families Anthribidae, Bruchidae and Curculionidae (including, but not limited to: Anthonomus grandis Boheman (boll weevil); Lissorhoptrus oryzophilus Kuschel (rice water weevil); Sitophilus granarius Linnaeus (granary weevil); S. oryzae Linnaeus (rice weevil); Hypera punctata Fabricius (clover leaf weevil); Cylindrocopturus adspersus LeConte (sunflower stem weevil); Smicronyx fulvus LeConte (red sunflower seed weevil); S. sordidus LeConte (gray sunflower seed weevil); Sphenophorus maidis Chittenden (maize billbug)); flea beetles, cucumber beetles, rootworms, leaf beetles, potato beetles and leafminers in the family Chrysomelidae (including, but not limited to: Leptinotarsa decemlineata Say (Colorado potato beetle); Diabrotica virgifera virgifera LeConte (western corn rootworm); D. barberi Smith and Lawrence (northern corn rootworm); D. undecimpunctata howardi Barber (southern corn rootworm); Chaetocnema pulicaria Melsheimer (corn flea beetle); Phyllotreta cruciferae Goeze (Crucifer flea beetle); Phyllotreta striolata (stripped flea beetle); Colaspis brunnea Fabricius (grape colaspis); Oulema melanopus Linnaeus (cereal leaf beetle); Zygogramma exclamationis Fabricius (sunflower beetle)); beetles from the family Coccinellidae (including, but not limited to: Epilachna varivestis Mulsant (Mexican bean beetle)); chafers and other beetles from the family Scarabaeidae (including, but not limited to: Popillia japonica Newman (Japanese beetle); Cyclocephala borealis Arrow (northern masked chafer, white grub); C. immaculata Olivier (southern masked chafer, white grub); Rhizotrogus majalis Razoumowsky (European chafer); Phyllophaga crinita Burmeister (white grub); Ligyrus gibbosus De Geer (carrot beetle)); carpet beetles from the family Dermestidae; wireworms from the family Elateridae, Eleodes spp., Melanotus spp.; Conoderus spp.; Limonius spp.; Agriotes spp.; Ctenicera spp.; Aeolus spp.; bark beetles from the family Scolytidae and beetles from the family Tenebrionidae.

Adults and immatures of the order Diptera are of interest, including leafminers Agromyza parvicornis Loew (corn blotch leafminer); midges (including, but not limited to: Contarinia sorghicola Coquillett (sorghum midge); Mayetiola destructor Say (Hessian fly); Sitodiplosis mosellana Géhin (wheat midge); Neolasioptera murtfeldtiana Felt, (sunflower seed midge)); fruit flies (Tephritidae), Oscinella frit Linnaeus (fruit flies); maggots (including, but not limited to: Delia platura Meigen (seedcorn maggot); D. coarctata Fallen (wheat bulb fly) and other Delia spp., Meromyza americana Fitch (wheat stem maggot); Musca domestica Linnaeus (house flies); Fannia canicularis Linnaeus, F. femoralis Stein (lesser house flies); Stomoxys calcitrans Linnaeus (stable flies)); face flies, horn flies, blow flies, Chrysomya spp.; Phormia spp. and other muscoid fly pests, horse flies Tabanus spp.; bot flies Gastrophilus spp.; Oestrus spp.; cattle grubs Hypoderma spp.; deer flies Chrysops spp.; Melophagus ovinus Linnaeus (keds) and other Brachycera, mosquitoes Aedes spp.; Anopheles spp.; Culex spp.; black flies Prosimulium spp.; Simulium spp.; biting midges, sand flies, sciarids, and other Nematocera.

Included as insects of interest are adults and nymphs of the orders Hemiptera and Homoptera such as, but not limited to, adelgids from the family Adelgidae, plant bugs from the family Miridae, cicadas from the family Cicadidae, leafhoppers, Empoasca spp.; from the family Cicadellidae, planthoppers from the families Cixiidae, Flatidae, Fulgoroidea, Issidae and Delphacidae, treehoppers from the family Membracidae, psyllids from the family Psyllidae, whiteflies from the family Aleyrodidae, aphids from the family Aphididae, phylloxera from the family Phylloxeridae, mealybugs from the family Pseudococcidae, scales from the families Asterolecanidae, Coccidae, Dactylopiidae, Diaspididae, Eriococcidae Ortheziidae, Phoenicococcidae and Margarodidae, lace bugs from the family Tingidae, stink bugs from the family Pentatomidae, cinch bugs, Blissus spp.; and other seed bugs from the family Lygaeidae, spittlebugs from the family Cercopidae squash bugs from the family Coreidae and red bugs and cotton stainers from the family Pyrrhocoridae.

Agronomically important members from the order Homoptera further include, but are not limited to: Acyrthisiphon pisum Harris (pea aphid); Aphis craccivora Koch (cowpea aphid); A. fabae Scopoli (black bean aphid); A. gossypii Glover (cotton aphid, melon aphid); A. maidiradicis Forbes (corn root aphid); A. pomi De Geer (apple aphid); A. spiraecola Patch (spirea aphid); Aulacorthum solani Kaltenbach (foxglove aphid); Chaetosiphon fragaefolii Cockerell (strawberry aphid); Diuraphis noxia Kurdjumov/Mordvilko (Russian wheat aphid); Dysaphis plantaginea Paaserini (rosy apple aphid); Eriosoma lanigerum Hausmann (woolly apple aphid); Brevicoryne brassicae Linnaeus (cabbage aphid); Hyalopterus pruni Geoffroy (mealy plum aphid); Lipaphis erysimi Kaltenbach (turnip aphid); Metopolophium dirrhodum Walker (cereal aphid); Macrosiphum euphorbiae Thomas (potato aphid); Myzus persicae Sulzer (peach-potato aphid, green peach aphid); Nasonovia ribisnigri Mosley (lettuce aphid); Pemphigus spp. (root aphids and gall aphids); Rhopalosiphum maidis Fitch (corn leaf aphid); R. padi Linnaeus (bird cherry-oat aphid); Schizaphis graminum Rondani (greenbug); Sipha flava Forbes (yellow sugarcane aphid); Sitobion avenae Fabricius (English grain aphid); Therioaphis maculata Buckton (spotted alfalfa aphid); Toxoptera aurantii Boyer de Fonscolombe (black citrus aphid) and T. citricida Kirkaldy (brown citrus aphid); Melanaphis sacchari (sugarcane aphid); Adelges spp. (adelgids); Phylloxera devastatrix Pergande (pecan phylloxera); Bemisia tabaci Gennadius (tobacco whitefly, sweetpotato whitefly); B. argentifolii Bellows & Perring (silverleaf whitefly); Dialeurodes citri Ashmead (citrus whitefly); Trialeurodes abutiloneus (bandedwinged whitefly) and T. vaporariorum Westwood (greenhouse whitefly); Empoasca fabae Harris (potato leafhopper); Laodelphax striatellus Fallen (smaller brown planthopper); Macrolestes quadrilineatus Forbes (aster leafhopper); Nephotettix cinticeps Uhler (green leafhopper); N. nigropictus Stål (rice leafhopper); Nilaparvata lugens Stål (brown planthopper); Peregrinus maidis Ashmead (corn planthopper); Sogatella furcifera Horvath (white-backed planthopper); Sogatodes orizicola Muir (rice delphacid); Typhlocyba pomaria McAtee (white apple leafhopper); Erythroneoura spp. (grape leafhoppers); Magicicada septendecim Linnaeus (periodical cicada); Icerya purchasi Maskell (cottony cushion scale); Quadraspidiotus perniciosus Comstock (San Jose scale); Planococcus citri Risso (citrus mealybug); Pseudococcus spp. (other mealybug complex); Cacopsylla pyricola Foerster (pear psylla); Trioza diospyri Ashmead (persimmon psylla).

Agronomically important species of interest from the order Hemiptera include, but are not limited to: Acrosternum hilare Say (green stink bug); Anasa tristis De Geer (squash bug); Blissus leucopterus leucopterus Say (chinch bug); Corythuca gossypii Fabricius (cotton lace bug); Cyrtopeltis modesta Distant (tomato bug); Dysdercus suturellus Herrich-Schäffer (cotton stainer); Euschistus servus Say (brown stink bug); E. variolarius Palisot de Beauvois (one-spotted stink bug); Graptostethus spp. (complex of seed bugs); Leptoglossus corculus Say (leaf-footed pine seed bug); Lygus lineolaris Palisot de Beauvois (tarnished plant bug); L. hesperus Knight (Western tarnished plant bug); L. pratensis Linnaeus (common meadow bug); L. rugulipennis Poppius (European tarnished plant bug); Lygocoris pabulinus Linnaeus (common green capsid); Nezara viridula Linnaeus (southern green stink bug); Oebalus pugnax Fabricius (rice stink bug); Oncopeltus fasciatus Dallas (large milkweed bug); Pseudatomoscelis seriatus Reuter (cotton fleahopper).

Furthermore, embodiments may be effective against Hemiptera such, Calocoris norvegicus Gmelin (strawberry bug); Orthops campestris Linnaeus; Plesiocoris rugicollis Fallen (apple capsid); Cyrtopeltis modestus Distant (tomato bug); Cyrtopeltis notatus Distant (suckfly); Spanagonicus albofasciatus Reuter (whitemarked fleahopper); Diaphnocoris chlorionis Say (honeylocust plant bug); Labopidicola allii Knight (onion plant bug); Pseudatomoscelis seriatus Reuter (cotton fleahopper); Adelphocoris rapidus Say (rapid plant bug); Poecilocapsus lineatus Fabricius (four-lined plant bug); Nysius ericae Schilling (false chinch bug); Nysius raphanus Howard (false chinch bug); Nezara viridula Linnaeus (Southern green stink bug); Eurygaster spp.; Coreidae spp.; Pyrrhocoridae spp.; Tinidae spp.; Blostomatidae spp.; Reduviidae spp. and Cimicidae spp.

Also included are adults and larvae of the order Acari (mites) such as Aceria tosichella Keifer (wheat curl mite); Petrobia latens Müller (brown wheat mite); spider mites and red mites in the family Tetranychidae, Panonychus ulmi Koch (European red mite); Tetranychus urticae Koch (two spotted spider mite); (T. mcdanieli McGregor (McDaniel mite); T. cinnabarinus Boisduval (carmine spider mite); T. turkestani Ugarov & Nikolski (strawberry spider mite); flat mites in the family Tenuipalpidae, Brevipalpus lewisi McGregor (citrus flat mite); rust and bud mites in the family Eriophyidae and other foliar feeding mites and mites important in human and animal health, i.e., dust mites in the family Epidermoptidae, follicle mites in the family Demodicidae, grain mites in the family Glycyphagidae, ticks in the order Ixodidae. Ixodes scapularis Say (deer tick); I. holocyclus Neumann (Australian paralysis tick); Dermacentor variabilis Say (American dog tick); Amblyomma americanum Linnaeus (lone star tick) and scab and itch mites in the families Psoroptidae, Pyemotidae and Sarcoptidae.

Insect pests of the order Thysanura are of interest, such as Lepisma saccharina Linnaeus (silverfish); Thermobia domestica Packard (firebrat).

Additional arthropod pests covered include: spiders in the order Araneae such as Loxosceles reclusa Gertsch and Mulaik (brown recluse spider) and the Latrodectus mactans Fabricius (black widow spider) and centipedes in the order Scutigeromorpha such as Scutigera coleoptrata Linnaeus (house centipede).

Insect pest of interest include the superfamily of stink bugs and other related insects including but not limited to species belonging to the family Pentatomidae (Nezara viridula, Halyomorpha halys, Piezodorus guildini, Euschistus servus, Acrosternum hilare, Euschistus heros, Euschistus tristigmus, Acrosternum hilare, Dichelops furcatus, Dichelops melacanthus, and Bagrada hilaris (Bagrada Bug)), the family Plataspidae (Megacopta cribraria—Bean plataspid) and the family Cydnidae (Scaptocoris castanea—Root stink bug) and Lepidoptera species including but not limited to: diamond-back moth, e.g., Helicoverpa zea Boddie; soybean looper, e.g., Pseudoplusia includens Walker and velvet bean caterpillar e.g., Anticarsia gemmatalis Hübner.

Methods for measuring pesticidal activity are well known in the art. See, for example, Czapla and Lang, (1990) J. Econ. Entomol. 83:2480-2485; Andrews, et al., (1988) Biochem. J. 252:199-206; Marrone, et al., (1985) J. of Economic Entomology 78:290-293 and U.S. Pat. No. 5,743,477. Generally, the pesticide is mixed and used in feeding assays. See, for example Marrone, et al., (1985) J. of Economic Entomology 78:290-293. Such assays can include contacting plants with one or more pests and determining the plant's ability to survive and/or cause the death of the pests.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

In some embodiments, a depsipeptide biosynthesis gene polypeptide comprises an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of any one of SEQ ID NOs: 109-208. In some embodiments, a nucleic acid sequence encoding a depsipeptide biosynthesis gene polypeptide comprises an polynucletoide sequence having at least about 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of any one of SEQ ID NOs: 9-108.

As used herein, the term “plant” refers to all plants, plant parts, seed, and plant populations, such as desirable and undesirable wild plants, cultivars, transgenic plants, and plant varieties (whether or not protectable by plant variety or plant breeder's rights). Cultivars and plant varieties can be plants obtained by conventional propagation and breeding methods that can be assisted or supplemented by one or more biotechnological methods such as by use of double haploids, protoplast fusion, random and directed mutagenesis, molecular or genetic markers or by bioengineering and genetic engineering methods.

The embodiments disclosed herein may generally be used for any plant species, including, but not limited to, monocots and dicots. Examples of plants of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables ornamentals, and conifers.

As used herein, the term “plant parts” refers to all above ground and below ground parts and organs of plants such as shoot, leaf, blossom and root, whereby for example leaves, needles, stems, branches, blossoms, fruiting bodies, fruits and seeds, as well as roots, tubers, corms and rhizomes are included. Crops and vegetative and generative propagating material, for example, cuttings, corms, rhizomes, tubers, runners and seeds are also plant parts.

As used herein, the term “viable” refers to a microbial cell, propagule, or spore that is metabolically active or able to differentiate. Thus, propagules, such as spores, are “viable” when they are dormant and capable of germinating.

The embodiments disclosed herein relate to a Pseudomonas chlororaphis strain SS532D1 (NRRL Deposit No. B-67638), a Pseudomonas chlororaphis strain SSP459B9-3 (NRRL Deposit No. B-67639), a Burkholderia rinojensis strain JH59178-1 (NRRL Deposit No. B-67640), a Chromobacterium haemolyticum strain JH91791-1 (NRRL Deposit No. B-67642), a Chromobacterium haemolyticum strain PMCJ4191H4-1 (NRRL Deposit No. B-67644), a Chromobacterium haemolyticum strain JH97285-1 (NRRL Deposit No. B-67641), or a Chromobacterium haemolyticum strain PMC3591F10-1 (NRRL Deposit No. B-67643); a fermentate produced from a growth medium comprising a Pseudomonas chlororaphis strain SS532D1 (NRRL Deposit No. B-67638), a Pseudomonas chlororaphis strain SSP459B9-3 (NRRL Deposit No. B-67639), a Burkholderia rinojensis strain JH59178-1 (NRRL Deposit No. B-67640), a Chromobacterium haemolyticum strain JH91791-1 (NRRL Deposit No. B-67642), a Chromobacterium haemolyticum strain PMCJ4191H4-1 (NRRL Deposit No. B-67644), a Chromobacterium haemolyticum strain JH97285-1 (NRRL Deposit No. B-67641), or a Chromobacterium haemolyticum strain PMC3591F10-1 (NRRL Deposit No. B-67643), and/or a depsipeptide in an effective amount to achieve an effect of inhibit growth of a plant pathogen, pest or insect. In one embodiment the bacterial strain disclosed herein, or a progeny, mutant, or variant thereof; fermentate produced from a strain disclosed herein progeny, mutant, or variant thereof; and/or depsipeptide compositions and methods find use in inhibiting, controlling, or killing a pathogen, pest, or insect, including, but is not limited to, fungi, pathogenic fungi, bacteria, mites, ticks, pathogenic microorganisms, and nematodes, as well as insects from the orders Coleoptera, Diptera, Hymenoptera, Lepidoptera, Mallophaga, Homoptera, Hemiptera Orthroptera, Thysanoptera, Dermaptera, Isoptera, Anoplura, Siphonaptera, Trichoptera, etc., particularly Coleoptera, including but not limited to Diabrotica virgifera virgifera, Diabrotica undecimpunctata howardi, and Diabrotica barberi, and for producing compositions with pesticidal activity.

The Pseudomonas chlororaphis strain SS532D1 (NRRL Deposit No. B-67638), Pseudomonas chlororaphis strain SSP459B9-3 (NRRL Deposit No. B-67639), Burkholderia rinojensis strain JH59178-1 (NRRL Deposit No. B-67640), Chromobacterium haemolyticum strain JH91791-1 (NRRL Deposit No. B-67642), Chromobacterium haemolyticum strain PMCJ4191H4-1 (NRRL Deposit No. B-67644), Chromobacterium haemolyticum strain JH97285-1 (NRRL Deposit No. B-67641), and Chromobacterium haemolyticum strain PMC3591F10-1 (NRRL Deposit No. B-67643) were deposited on Jul. 20, 2018 at the Agricultural Research Service Culture Collection (NRRL), 1815 North University Street, Peoria, Ill., 61604. The deposits were made under the provisions of the Budapest Treaty on the International Recognition of the Deposit of Microorganisms for the Purposes of Patent Procedure. Further, these deposits will be maintained under the terms of the Budapest Treaty on the International Recognition of the Deposit of Microorganisms for the Purposes of Patent Procedure. Access to these deposits will be available during the pendency of the application to the Commissioner of Patents and Trademarks and persons determined by the Commissioner to be entitled thereto upon request. Upon allowance of any claims in the application, the Applicant will make available to the public, pursuant to 37 C.F.R. § 1.808, sample(s) of the deposits. The deposits will be maintained in the NRRL depository, which is a public depository, for a period of 30 years, or 5 years after the most recent request, or for the enforceable life of the patent, whichever is longer, and will be replaced if it becomes nonviable during that period. Additionally, Applicant has satisfied all the requirements of 37 C.F.R. §§ 1.801-1.809, including providing an indication of the viability of the sample upon deposit.

Some embodiments relate to compositions comprising or consisting of or consisting essentially of a bacterial strain disclosed herein, or a progeny, mutant, or variant thereof, a fermentate produced from a strain disclosed herein progeny, mutant, or variant thereof, and/or a depsipeptide. In one embodiment, the compositions are biologically pure cultures of the strain disclosed herein.

Some embodiments relate to a composition comprising a bacterial strain disclosed herein, or a progeny, mutant, or variant thereof, a fermentate produced from a strain disclosed herein progeny, mutant, or variant thereof, and/or a depsipeptide disclosed herein and one or more compounds or agents selected from the group consisting of: agrochemically active compounds, biocontrol agents, lipo-chitooligosaccharide compounds (LCOs), isoflavones, quinazolines, insecticidal compounds, azolopyrimidinylamines, polymeric compounds, ionic compound, substituted thiophenes, substituted dithiines, fluopyramm, enaminocarbonyl compounds, strigolactone compound, and dithiino-tetracarboximide compounds.

A further embodiment relates to the use of a first composition comprising a bacterial strain disclosed herein, or a progeny, mutant, or variant thereof, a fermentate produced from a strain disclosed herein progeny, mutant, or variant thereof, and/or a depsipeptide disclosed herein and a second composition comprising one or more compounds or agents selected from the group consisting of: agrochemically active compounds, biocontrol agents, lipo-chitooligosaccharide compounds (LCOs), isoflavones, quinazolines, insecticidal compound, azolopyrimidinylamine, polymeric compounds, ionic compound, substituted thiophenes, substituted dithiines, fluopyramm, enaminocarbonyl compounds, strigolactone compound, and dithiino-tetracarboximide compounds.

In one embodiment, the disclosure relates to a composition comprising a bacterial strain disclosed herein, or a progeny, mutant, or variant thereof, a fermentate produced from a strain disclosed herein progeny, mutant, or variant thereof, and/or a depsipeptide disclosed herein and one or more biocontrol agents, As used herein, the term “biocontrol agent” (“BCA”) includes bacteria, fungi or yeasts, protozoans, viruses, entomopathogenic nematodes, and botanical extracts, or products produced by microorganisms including proteins or secondary metabolite, and inoculants that have one or both of the following characteristics: (1) inhibits or reduces plant infestation and/or growth of pathogens, pests, or insects, including but not limited to pathogenic fungi, bacteria, and nematodes, as well as arthropod pests such as insects, arachnids, chilopods, diplopods, or that inhibits plant infestation and/or growth of a combination of plant pathogens, pests, or insects; (2) improves plant performance; (3) improves plant yield; (4) improves plant vigor; and (5) improves plant health.

In one embodiment, the disclosure relates to a composition comprising a bacterial strain disclosed herein, or a progeny, mutant, or variant thereof, a fermentate produced from a strain disclosed herein progeny, mutant, or variant thereof, and/or a depsipeptide disclosed herein and one or more agrochemically active compounds. Agrochemically active compounds are substances that are or may be used for treating a seed, a plant, plant part, or the environment of the seed or plant or plant part including but not limited to fungicides, bactericides, insecticides, acaricides, nematicides, molluscicides, safeners, plant growth regulators, plant nutrients, chemical entities with a known mechanism of action, additional microorganisms, and biocontrol agents.

In another embodiment, the disclosure relates to a first composition comprising a bacterial strain disclosed herein, or a progeny, mutant, or variant thereof, a fermentate produced from a strain disclosed herein progeny, mutant, or variant thereof, and/or a depsipeptide and a second composition comprising one or more agrochemically active compounds, wherein the first and second composition may inhibit plant pathogens, pests, or insects and/or improve plant performance.

In one embodiment, the first and second compositions can be applied at the same time to a seed, a plant, plant part, or the environment of the plant. In another embodiment, the first composition can be applied to the seed followed by application of the second composition to the seed. In yet another embodiment, the second composition can be applied to the seed followed by application of the first composition to the seed.

In another embodiment, the first composition can be applied to the plant or plant part followed by application of the second composition to the plant or plant part. In yet another embodiment, the second composition can be applied to the plant or plant part followed by application of the first composition to the plant or plant part.

In another embodiment, the first composition can be applied to the seed and the second composition applied to the plant or plant part, In yet another embodiment, the second composition can be applied to the seed and the first composition applied to the plant or plant part.

In another embodiment, the first composition may be planted on or near the seed in a field. In yet another embodiment, the second composition can be applied to the seed and the first composition applied to the plant or plant part.

In one embodiment, the disclosure relates to the use of a bacterial strain disclosed herein, or a progeny, mutant, or variant thereof, a fermentate produced from a strain disclosed herein progeny, mutant, or variant thereof, and/or a depsipeptide disclosed herein with a composition comprising an insecticidal protein from Pseudomonas sp. such as PSEEN3174 (Monalysin; (2011) PLoS Pathogens 7:1-13); from Pseudomonas protegens strain CHA0 and Pf-5 (previously fluorescens) (Pechy-Tarr, (2008) Environmental Microbiology 10:2368-2386; GenBank Accession No. EU400157); from Pseudomonas Taiwanensis (Liu, et al., (2010) J. Agric. Food Chem., 58:12343-12349) and from Pseudomonas pseudoalcligenes (Zhang, et al., (2009) Annals of Microbiology 59:45-50 and Li, et al., (2007) Plant Cell Tiss. Organ Cult. 89:159-168); insecticidal proteins from Photorhabdus sp. and Xenorhabdus sp. (Hinchliffe, et al., (2010) The Open Toxicology Journal, 3:101-118 and Morgan, et al., (2001) Applied and Envir. Micro. 67:2062-2069); U.S. Pat. Nos. 6,048,838, and 6,379,946; a PIP-1 polypeptide of U.S. Pat. No. 9,688,730; an AfIP-1A and/or AfIP-1B polypeptide of U.S. Pat. No. 9,475,847; a PIP-47 polypeptide of US Publication Number US20160186204; an IPD045 polypeptide, an IPD064 polypeptide, an IPD074 polypeptide, an IPD075 polypeptide, and an IPD077 polypeptide of PCT Publication Number WO 2016/114973; an IPD080 polypeptide of PCT Serial Number PCT/US17/56517; an IPD078 polypeptide, an IPD084 polypeptide, an IPD085 polypeptide, an IPD086 polypeptide, an IPD087 polypeptide, an IPD088 polypeptide, and an IPD089 polypeptide of Serial Number PCT/US17/54160; PIP-72 polypeptide of US Patent Publication Number US20160366891; a PtIP-50 polypeptide and a PtIP-65 polypeptide of US Publication Number US20170166921; an IPD098 polypeptide, an IPD059 polypeptide, an IPD108 polypeptide, an IPD109 polypeptide of U.S. Ser. No. 62/521,084; a PtIP-83 polypeptide of US Publication Number US20160347799; a PtIP-96 polypeptide of US Publication Number US20170233440; an IPD079 polypeptide of PCT Publication Number WO2017/23486; an IPD082 polypeptide of PCT Publication Number WO 2017/105987, an IPD090 polypeptide of Serial Number PCT/US17/30602, an IPD093 polypeptide of U.S. Ser. No. 62/434,020; an IPD103 polypeptide of Serial Number PCT/US17/39376; an IPD101 polypeptide of U.S. Ser. No. 62/438,179; an IPD121 polypeptide of US Serial Number U.S. 62/508,514; and δ-endotoxins including, but not limited to, the Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry 28, Cry 29, Cry 30, Cry31, Cry32, Cry33, Cry34, Cry35,Cry36, Cry37, Cry38, Cry39, Cry40, Cry41, Cry42, Cry43, Cry44, Cry45, Cry 46, Cry47, Cry49, Cry 51 and Cry55 classes of δ-endotoxin genes and the B. thuringiensis cytolytic Cyt1 and Cyt2 genes. Other Cry proteins are well known to one skilled in the art (see, Crickmore, et al., “Bacillus thuringiensis toxin nomenclature” (2011), at lifesci.sussex.ac.uk/home/Neil_Crickmore/Bt/ which can be accessed on the world-wide web using the “www” prefix). The insecticidal activity of Cry proteins is well known to one skilled in the art (for review, see, van Frannkenhuyzen, (2009) J. Invert. Path. 101:1-16).

In one embodiment the composition comprises a silencing element of one or more polynucleotides of interest resulting in suppression of one or more target pathogen, pest, or insect polypeptides. By “silencing element” is it intended to mean a polynucleotide which when contacted by or ingested by a pest, is capable of reducing or eliminating the level or expression of a target polynucleotide or the polypeptide encoded thereby. The silencing element employed can reduce or eliminate the expression level of the target sequence by influencing the level of the target RNA transcript or, alternatively, by influencing translation and thereby affecting the level of the encoded polypeptide. Silencing elements may include, but are not limited to, a sense suppression element, an antisense suppression element, a double stranded RNA, a siRNA, an amiRNA, a miRNA, or a hairpin suppression element.

Nucleic acid molecules including silencing elements for targeting the vacuolar ATPase H subunit, useful for controlling a coleopteran pest population and infestation as described in US Patent Application Publication 2012/0198586. PCT Publication WO 2012/055982 describes ribonucleic acid (RNA or double stranded RNA) that inhibits or down regulates the expression of a target gene that encodes: an insect ribosomal protein such as the ribosomal protein L19, the ribosomal protein L40 or the ribosomal protein S27A; an insect proteasome subunit such as the Rpn6 protein, the Pros 25, the Rpn2 protein, the proteasome beta 1 subunit protein or the Pros beta 2 protein; an insect β-coatomer of the COPI vesicle, the γ-coatomer of the COPI vesicle, the β′-coatomer protein or the ζ-coatomer of the COPI vesicle; an insect Tetraspanine 2 A protein which is a putative transmembrane domain protein; an insect protein belonging to the actin family such as Actin 5C; an insect ubiquitin-5E protein; an insect Sec23 protein which is a GTPase activator involved in intracellular protein transport; an insect crinkled protein which is an unconventional myosin which is involved in motor activity; an insect crooked neck protein which is involved in the regulation of nuclear alternative mRNA splicing; an insect vacuolar H+-ATPase G-subunit protein and an insect Tbp-1 such as Tat-binding protein. PCT publication WO 2007/035650 describes ribonucleic acid (RNA or double stranded RNA) that inhibits or down regulates the expression of a target gene that encodes Snf7. US Patent Application publication 2011/0054007 describes polynucleotide silencing elements targeting RPS10. US Patent Application publication 2014/0275208 describes polynucleotide silencing elements targeting RyanR and PAT3. US Patent Application Publications 2012/029750, US 20120297501, and 2012/0322660 describe interfering ribonucleic acids (RNA or double stranded RNA) that functions upon uptake by an insect pest species to down-regulate expression of a target gene in said insect pest, wherein the RNA comprises at least one silencing element wherein the silencing element is a region of double-stranded RNA comprising annealed complementary strands, one strand of which comprises or consists of a sequence of nucleotides which is at least partially complementary to a target nucleotide sequence within the target gene. US Patent Application Publication 2012/0164205 describe potential targets for interfering double stranded ribonucleic acids for inhibiting invertebrate pests including: a Chd3 Homologous Sequence, a Beta-Tubulin Homologous Sequence, a 40 kDa V-ATPase Homologous Sequence, a EF1α Homologous Sequence, a 26S Proteosome Subunit p28 Homologous Sequence, a Juvenile Hormone Epoxide Hydrolase Homologous Sequence, a Swelling Dependent Chloride Channel Protein Homologous Sequence, a Glucose-6-Phosphate 1-Dehydrogenase Protein Homologous Sequence, an Act42A Protein Homologous Sequence, a ADP-Ribosylation Factor 1 Homologous Sequence, a Transcription Factor IIB Protein Homologous Sequence, a Chitinase Homologous Sequences, a Ubiquitin Conjugating Enzyme Homologous Sequence, a Glyceraldehyde-3-Phosphate Dehydrogenase Homologous Sequence, an Ubiquitin B Homologous Sequence, a Juvenile Hormone Esterase Homolog, and an Alpha Tubulin Homologous Sequence.

Some embodiments comprise an additional component, which may be a carrier, an adjuvant, a solubilizing agent, a suspending agent, a diluent, an oxygen scavenger, an antioxidant, a food material, an anti-contaminant agent, or combinations thereof.

In another embodiment, the additional component(s) may be required for the application to which the strain or composition is to be utilized. For example, if the strain or composition is to be utilized on, or in, an agricultural product, the additional component(s) may be an agriculturally acceptable carrier, excipient, or diluent. Likewise, if the strain or composition is to be utilized on, or in, a foodstuff the additional component(s) may be an edible carrier, excipient or diluent.

In one aspect, the one or more additional component(s) is a carrier, excipient, or diluent. “Carriers” or “vehicles” mean materials suitable for compound administration and include any such material known in the art such as, for example, any liquid, gel, solvent, liquid diluent, solubilizer, or the like, which is non-toxic and does not interact with any components of the composition in a deleterious manner.

Examples of nutritionally acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

Examples of excipients include but are not limited to: microcrystalline cellulose and other celluloses, lactose, sodium citrate, calcium carbonate, dibasic calcium phosphate, glycine, starch, milk sugar, and high molecular weight polyethylene glycols.

Examples of diluents include but are not limited to: water, ethanol, propylene glycol and glycerin, and combinations thereof.

The other components may be used simultaneously (e.g. when they are in admixture together or even when they are delivered by different routes) or sequentially (e.g. they may be delivered by different routes).

The composition or its diluent may also contain chelating agents such as EDTA, citric acid, tartaric acid, etc. Moreover, the composition or its diluent may contain active agents selected from fatty acids esters, such as mono-and diglycerides, non-ionic surfactants, such as polysorbates, phospholipids, etc. Emulsifiers may enhance the stability of the composition, especially after dilution.

The bacterial strain disclosed herein, or a progeny, mutant, or variant thereof, a fermentate produced from a strain disclosed herein progeny, mutant, or variant thereof, and/or a depsipeptide may be used in any suitable form—whether when alone or when present in a composition. The compositions may be formulated in any suitable way to ensure that the composition comprises an active compound(s) of interest.

The bacterial strain disclosed herein, or a progeny, mutant, or variant thereof, a fermentate produced from a strain disclosed herein progeny, mutant, or variant thereof, and/or a depsipeptide compositions thereof may be in the form of a dry powder that can be sprinkled on or mixed in with a product. The compositions in the form of a dry powder may include an additive such as microcrystalline cellulose, gum tragacanth, gelatin, starch, lactose, alginic acid, Primogel, or corn starch (which can be used as a disintegrating agent).

In yet another embodiment, a bacterial strain disclosed herein, or a progeny, mutant, or variant thereof, a fermentate produced from a strain disclosed herein progeny, mutant, or variant thereof, and/or a depsipeptide compositions disclosed herein can be a spray-dried fermentate re-suspended in H₂O to a percentage selected from the following: 0.05-1, 1-3, 3-5, 5-7, 7-10, 10-15, 15-20, and greater than 20%. In another embodiment, one or more than one clarification step(s) can be performed prior to spray-drying.

In one embodiment, the compositions disclosed herein can comprise concentrated, dried propagules, from the strain disclosed herein. In one embodiment, compositions can be in the range of 1×10³ to 1×10¹³ CFU/g.

In one embodiment, the compositions disclosed herein can be applied in wet or partially or completely desiccated form or in slurry, gel, or other form.

In at least some embodiments, the compositions disclosed herein can be freeze-dried or lypholized. In at least some embodiments, the compositions can be mixed with a carrier. The carrier includes but is not limited to whey, maltodextrin, sucrose, dextrose, limestone (calcium carbonate), rice hulls, yeast culture, dried starch, clay, and sodium silico aluminate. The compositions can also be used with or without preservatives and in concentrated, un-concentrated, or diluted form. In one embodiment, the compositions can be in the form of a pellet or a biologically pure pellet.

The compositions described herein can be added to one or more carrier. Where used, the carrier(s) and the compositions can be added to a ribbon or paddle mixer and mixed for about 15 minutes, although the timing can be increased or decreased. The components are blended such that a uniform mixture of the culture and carrier(s) is produced. The final product is preferably a dry, flowable powder.

In one embodiment, the compositions may be formulated as a liquid, a dry powder, or a granule. The dry powder or granules may be prepared by means known to those skilled in the art, such as, in top-spray fluid bed coater, in a bottom spray Wurster, or by drum granulation (e.g. high sheer granulation), extrusion, pan coating or in a micro-ingredients mixer.

In another embodiment, the compositions disclosed herein may be provided as a spray-dried or freeze-dried powder.

In yet another embodiment, the compositions are in a liquid formulation. Such liquid consumption may contain one or more of the following: a buffer, salt, sorbitol, and/or glycerol.

In one embodiment, the compositions disclosed herein may be formulated with at least one physiologically acceptable carrier selected from at least one of maltodextrin, calcined (illite) clay, limestone (calcium carbonate), cyclodextrin, wheat or a wheat component, sucrose, starch, Na₂SO₄, Talc, PVA, sorbitol, benzoate, sorbiate, glycerol, sucrose, propylene glycol, 1,3-propane diol, glucose, parabens, sodium chloride, citrate, acetate, phosphate, calcium, metabisulfite, formate and mixtures thereof.

In one embodiment, the compositions disclosed herein may be formulated by encapsulation technology to improve stability and as a way to protect the compositions from seed applications. In one embodiment the encapsulation technology may comprise a bead polymer for timed release of the compositions over time. In one embodiment, the encapsulated compositions may be applied in a separate application of beads in-furrow to the seeds. In another embodiment, the encapsulated compositions may be co-applied along with seeds simultaneously.

The coating agent usable for the sustained release microparticles of an encapsulation embodiment may be a substance which is useful for coating the microgranular form with the substance to be supported thereon. Any coating agent which can form a coating difficultly permeable for the supported substance may be used in general, without any particular limitation. For example, higher saturated fatty acid, wax, thermoplastic resin, thermosetting resin and the like may be used.

Examples of useful higher saturated fatty acid include stearic acid, zinc stearate, stearic acid amide and ethylenebis-stearic acid amide; those of wax include synthetic waxes such as polyethylene wax, carbon wax, Hoechst wax, and fatty acid ester; natural waxes such as carnauba wax, bees wax and Japan wax; and petroleum waxes such as paraffin wax and petrolatum. Examples of thermoplastic resin include polyolefins such as polyethylene, polypropylene, polybutene and polystyrene; vinyl polymers such as polyvinyl acetate, polyvinyl chloride, polyvinylidene chloride, polyacrylic acid, polymethacrylic acid, polyacrylate and polymethacrylate; diene polymers such as butadiene polymer, isoprene polymer, chloroprene polymer, butadiene-styrene copolymer, ethylene-propylene-diene copolymer, styrene-isoprene copolymer, MMA-butadiene copolymer and acrylonitrile-butadiene copolymer; polyolefin copolymers such as ethylene-propylene copolymer, butene-ethylene copolymer, butene-propylene copolymer, ethylene-vinyl acetate copolymer, ethylene-acrylic acid copolymer, styreneacrylic acid copolymer, ethylene-methacrylic acid copolymer, ethylene-methacrylic ester copolymer, ethylene-carbon monoxide copolymer, ethylene-vinyl acetate-carbon monoxide copolymer, ethylene-vinyl acetate-vinyl chloride copolymer and ethylene-vinyl acetate-acrylic copolymer; and vinyl chloride copolymers such as vinyl chloride-vinyl acetate copolymer and vinylidene chloride-vinyl chloride copolymer. Examples of thermosetting resin include polyurethane resin, epoxy resin, alkyd resin, unsaturated polyester resin, phenolic resin, urea-melamine resin, urea resin and silicone resin. Of those, thermoplastic acrylic ester resin, butadienestyrene copolymer resin, thermosetting polyurethane resin and epoxy resin are preferred, and among the preferred resins, particularly thermosetting polyurethane resin is preferred. These coating agents can be used either singly or in combination of two or more kinds.

In one embodiment, the compositions may include a seed, a part of a seed, a plant, or a plant part.

All plants, plant parts, seeds or soil may be treated in accordance with the compositions and methods disclosed herein. The compositions disclosed herein may include a plant, a plant part, a seed, a seed part, or soil. The compositions and methods disclosed herein may be applied to the seed, the plant or plant parts, the fruit, or the soil in which the plants grow.

Some embodiments relate to a method for reducing plant pathogen, pest, or insect damage to a plant or plant part comprising: (a) treating a seed with a composition disclosed herein prior to planting. In another embodiment, the method further comprises: (b) treating a plant part obtained from the seed with a composition disclosed herein. The composition used in step (a) may be the same or different than the composition used in step (b).

Some embodiments relate to a method for reducing plant pathogen, pest, or insect damage to a plant or plant part comprising: (a) treating the soil surrounding a seed or plant a bacterial strain disclosed herein, or a progeny, mutant, or variant thereof, a fermentate produced from a strain disclosed herein progeny, mutant, or variant thereof, and/or a depsipeptide. In another embodiment, the method further compri ses: (b) treating a plant part with a bacterial strain disclosed herein, or a progeny, mutant, or variant thereof, a fermentate produced from a strain disclosed herein progeny, mutant, or variant thereof, and/or a depsipeptide disclosed herein. The bacterial strain disclosed herein, or a progeny, mutant, or variant thereof, a fermentate produced from a strain disclosed herein progeny, mutant, or variant thereof, and/or a depsipeptide used in step (a) may be the same or different than a bacterial strain disclosed herein, or a progeny, mutant, or variant thereof, a fermentate produced from a strain disclosed herein progeny, mutant, or variant thereof, and/or a depsipeptide used in step (b).

Some embodiments relate to a method for reducing plant pathogen, pest, or insect damage to a plant or plant part comprising: (a) treating a seed prior to planting with a bacterial strain disclosed herein, or a progeny, mutant, or variant thereof, a fermentate produced from a strain disclosed herein progeny, mutant, or variant thereof, and/or a depsipeptide disclosed herein. In another embodiment, the method further comprises: (b) treating the soil surrounding the seed or plant with a bacterial strain disclosed herein, or a progeny, mutant, or variant thereof, a fermentate produced from a strain disclosed herein progeny, mutant, or variant thereof, and/or a depsipeptide disclosed herein. In still another embodiment, the method further comprises: (c) treating a plant part of a plant produced from the seed with a bacterial strain disclosed herein, or a progeny, mutant, or variant thereof, a fermentate produced from a strain disclosed herein progeny, mutant, or variant thereof, and/or a depsipeptide disclosed herein. The bacterial strain disclosed herein, or a progeny, mutant, or variant thereof, a fermentate produced from a strain disclosed herein progeny, mutant, or variant thereof, and/or a depsipeptide used in step (a) may be the same or different than the bacterial strain disclosed herein, or a progeny, mutant, or variant thereof, a fermentate produced from a strain disclosed herein progeny, mutant, or variant thereof, and/or a depsipeptide used in step (b). The bacterial strain disclosed herein, or a progeny, mutant, or variant thereof, a fermentate produced from a strain disclosed herein progeny, mutant, or variant thereof, and/or a depsipeptide used in step (a) may be the same or different than the bacterial strain disclosed herein, or a progeny, mutant, or variant thereof, a fermentate produced from a strain disclosed herein progeny, mutant, or variant thereof, and/or a depsipeptide used in step (c). The bacterial strain disclosed herein, or a progeny, mutant, or variant thereof, a fermentate produced from a strain disclosed herein progeny, mutant, or variant thereof, and/or a depsipeptide used in step (b) may be the same or different than the bacterial strain disclosed herein, or a progeny, mutant, or variant thereof, a fermentate produced from a strain disclosed herein progeny, mutant, or variant thereof, and/or a depsipeptide used in step (c).

In one embodiment, wild plant species and plant cultivars, or those obtained by conventional biological breeding, such as crossing or protoplast fusion, and parts thereof, can be treated with a bacterial strain disclosed herein, or a progeny, mutant, or variant thereof, a fermentate produced from a strain disclosed herein progeny, mutant, or variant thereof, and/or a depsipeptide disclosed herein. In another embodiment, transgenic plants and plant cultivars obtained by genetic engineering, and plant parts thereof, are treated with a bacterial strain disclosed herein, or a progeny, mutant, or variant thereof, a fermentate produced from a strain disclosed herein progeny, mutant, or variant thereof, and/or a depsipeptide disclosed herein.

In another embodiment, plants or plant cultivars (obtained by plant biotechnology methods such as genetic engineering or editing) that may be treated according to the strains, compositions and methods disclosed herein are herbicide-tolerant plants, i.e. plants made tolerant to one or more given herbicides. Such plants can be obtained either by genetic modification, or by selection of plants containing a mutation imparting such herbicide tolerance. Herbicide-resistant plants are for example glyphosate-tolerant plants, i.e. plants made tolerant to the herbicide glyphosate or salts thereof. Plants can be made tolerant to glyphosate through different means. For example, glyphosate-tolerant plants can be obtained by transforming the plant with a gene encoding the enzyme 5-enolpyruvylshilcimate-3-phosphate synthase (EPSPS).

Plants or plant cultivars (obtained by plant biotechnology methods such as genetic engineering or editing) that may also be treated are insect-resistant genetically modified plants, i.e. plants made resistant to attack by certain target insects. Such plants can be obtained by genetic transformation, or by selection of plants containing a mutation imparting such insect resistance.

In another embodiment, plants or plant cultivars (obtained by plant biotechnology methods such as genetic engineering) that may be treated according to the disclosure are tolerant to abiotic stresses. Such plants can be obtained by genetic transformation, or by selection of plants containing a mutation imparting such stress resistance.

In another embodiment, plants or plant cultivars (obtained by plant biotechnology methods such as genetic engineering or editing) that may be treated according to the disclosure are conventionally bred, by mutagenesis, or genetically engineered to contain a combination or stack of valuable traits, including but not limited to, herbicide tolerance, insect resistance, and abiotic stress tolerance.

The embodiments disclosed herein also apply to plant varieties which will be developed, or marketed, in the future and which have these genetic traits or traits to be developed in the future.

As used herein, applying a bacterial strain disclosed herein, or a progeny, mutant, or variant thereof, a fermentate produced from a strain disclosed herein progeny, mutant, or variant thereof, and/or a depsipeptide to a seed, a plant, or plant part includes contacting the seed, plant, or plant part directly and/or indirectly with the bacterial strain disclosed herein, or a progeny, mutant, or variant thereof, a fermentate produced from a strain disclosed herein progeny, mutant, or variant thereof, and/or a depsipeptide. In one embodiment, a bacterial strain disclosed herein, or a progeny, mutant, or variant thereof, a fermentate produced from a strain disclosed herein progeny, mutant, or variant thereof, and/or a depsipeptide may be directly applied as a spray, a rinse, or a powder, or any combination thereof.

As used herein, a spray refers to a mist of liquid particles that contain a bacterial strain disclosed herein, or a progeny, mutant, or variant thereof, a fermentate produced from a strain disclosed herein progeny, mutant, or variant thereof, and/or a depsipeptide of the present disclosure. In one embodiment, a spray may be applied to a plant or plant part while a plant or plant part is being grown. In another aspect, a spray may be applied to a plant or plant part while a plant or plant part is being fertilized. In another aspect, a spray may be applied to a plant or plant part while a plant or plant part is being harvested. In another aspect, a spray may be applied to a plant or plant part after a plant or plant part has been harvested. In another aspect, a spray may be applied to a plant or plant part while a plant or plant part is being processed. In another aspect, a spray may be applied to a plant or plant part while a plant or plant part is being packaged. In another aspect, a spray may be applied to a plant or plant part while a plant or plant part is being stored.

In another embodiment, a bacterial strain disclosed herein, or a progeny, mutant, or variant thereof, a fermentate produced from a strain disclosed herein progeny, mutant, or variant thereof, and/or a depsipeptide disclosed herein may be applied directly to a plant or plant part as a rinse. As used herein, a rinse is a liquid containing a bacterial strain disclosed herein, or a progeny, mutant, or variant thereof, a fermentate produced from a strain disclosed herein progeny, mutant, or variant thereof, and/or a depsipeptide disclosed herein. Such a rinse may be poured over a plant or plant part. A plant or plant part may also be immersed or submerged in the rinse, then removed and allowed to dry.

In another embodiment, a bacterial strain disclosed herein, or a progeny, mutant, or variant thereof, a fermentate produced from a strain disclosed herein progeny, mutant, or variant thereof, and/or a depsipeptide may be applied to a plant or plant part and may cover 50% of the surface area of a plant material. In another embodiment, a bacterial strain disclosed herein, or a progeny, mutant, or variant thereof, a fermentate produced from a strain disclosed herein progeny, mutant, or variant thereof, and/or a depsipeptide may be applied to a plant or plant part and may cover a percentage of the surface area of a plant material selected from the group consisting of: from 50% to about 95%, from 60% to about 95%, from 70% to about 95%, from 80% to about 95%, and from 90% to about 95%.

In another aspect, a bacterial strain disclosed herein, or a progeny, mutant, or variant thereof, a fermentate produced from a strain disclosed herein progeny, mutant, or variant thereof, and/or a depsipeptide may cover from about 20% to about 30%, from about 30% to about 40%, from about 40% to about 50%, from about 50% to about 60%, from about 60% to about 70%, from about 70% to about 80%, from about 80% to about 90%, from about 90% to about 95%, from about 95% to about 98%, from about 98% to about 99% or 100% of the surface area of a plant or plant part.

In another aspect, a bacterial strain disclosed herein, or a progeny, mutant, or variant thereof, a fermentate produced from a strain disclosed herein progeny, mutant, or variant thereof, and/or a depsipeptide disclosed herein may be applied directly to a plant or plant part as a powder. As used herein, a powder is a dry or nearly dry bulk solid composed of a large number of very fine particles that may flow freely when shaken or tilted. A dry or nearly dry powder composition disclosed herein preferably contains a low percentage of water, such as, for example, in various aspects, less than 5%, less than 2.5%, or less than 1% by weight.

In another aspect, a composition can be applied indirectly to the plant or plant part. For example, a plant or plant part having a bacterial strain disclosed herein, or a progeny, mutant, or variant thereof, a fermentate produced from a strain disclosed herein progeny, mutant, or variant thereof, and/or a depsipeptide already applied may be touching a second plant or plant part so that a bacterial strain disclosed herein, or a progeny, mutant, or variant thereof, a fermentate produced from a strain disclosed herein progeny, mutant, or variant thereof, and/or a depsipeptide rubs off on a second plant or plant part. In a further aspect, a bacterial strain disclosed herein, or a progeny, mutant, or variant thereof, a fermentate produced from a strain disclosed herein progeny, mutant, or variant thereof, and/or a depsipeptide may be applied using an applicator. In various aspects, an applicator may include, but is not limited to, a syringe, a sponge, a paper towel, or a cloth, or any combination thereof.

A contacting step may occur while a plant material is being grown, while a plant or plant part is being fertilized, while a plant or plant part is being harvested, after a plant or plant part has been harvested, while a plant or plant part is being processed, while a plant or plant part is being packaged, or while a plant or plant part is being stored in warehouse or on the shelf of a store.

In another embodiment, a bacterial strain disclosed herein, or a progeny, mutant, or variant thereof, a fermentate produced from a strain disclosed herein progeny, mutant, or variant thereof, and/or a depsipeptide disclosed herein may be a colloidal dispersion. A colloidal dispersion is a type of chemical mixture where one substance is dispersed evenly throughout another. Particles of the dispersed substance are only suspended in the mixture, unlike a solution, where they are completely dissolved within. This occurs because the particles in a colloidal dispersion are larger than in a solution—small enough to be dispersed evenly and maintain a homogenous appearance, but large enough to scatter light and not dissolve. Colloidal dispersions are an intermediate between homogeneous and heterogeneous mixtures and are sometimes classified as either “homogeneous” or “heterogeneous” based upon their appearance.

In one embodiment, the bacterial strain disclosed herein, or a progeny, mutant, or variant thereof, a fermentate produced from a strain disclosed herein progeny, mutant, or variant thereof, and/or a depsipeptide compositions and methods disclosed herein are suitable for use with a seed. In another embodiment, the the bacterial strain disclosed herein, or a progeny, mutant, or variant thereof, a fermentate produced from a strain disclosed herein progeny, mutant, or variant thereof, and/or a depsipeptide compositions and methods disclosed herein are suitable for use with a seed of one or more of any of the plants recited previously.

In still another embodiment, the bacterial strain disclosed herein, or a progeny, mutant, or variant thereof, a fermentate produced from a strain disclosed herein progeny, mutant, or variant thereof, and/or a depsipeptide compositions and methods disclosed herein can be used to treat transgenic or genetically modified or edited seed. A transgenic seed refers to the seed of plants containing at least one heterologous gene that allows the expression of a polypeptide or protein not naturally found in the plant. The heterologous gene in transgenic seed can originate, for example, from microorganisms of the species Bacillus, Rhizobium, Pseudomonas, Serratia, Trichoderma, Clavibacter, Glomus or Gliocladium.

In one embodiment, the seed is treated in a state in which it is sufficiently stable so that the treatment does not cause any damage. In general, treatment of the seed may take place at any point in time between harvesting and sowing. In one embodiment, the seed used is separated from the plant and freed from cobs, shells, stalks, coats, hairs or the flesh of the fruits. Thus, it is possible to use, for example, seed which has been harvested, cleaned and dried. Alternatively, it is also possible to use seed which, after drying, has been treated, for example, with water and then dried again.

In one embodiment, seed is treated with a bacterial strain disclosed herein, or a progeny, mutant, or variant thereof,a fermentate produced from a strain disclosed herein progeny, mutant, or variant thereof, and/or a depsipeptide compositions and methods disclosed herein in such a way that the germination of the seed is not adversely affected, or that the resulting plant is not damaged.

In one embodiment, a bacterial strain disclosed herein, or a progeny, mutant, or variant thereof, a fermentate produced from a strain disclosed herein progeny, mutant, or variant thereof, and/or a depsipeptide compositions disclosed herein may be applied directly to the seed. For example, the bacterial strain disclosed herein, or a progeny, mutant, or variant thereof, fermentate produced from a strain disclosed herein progeny, mutant, or variant thereof, and/or depsipeptide compositions disclosed herein may be applied without additional components and without having been diluted.

In another embodiment, a bacterial strain disclosed herein, or a progeny, mutant, or variant thereof, a fermentate produced from a strain disclosed herein progeny, mutant, or variant thereof, and/or a depsipeptide disclosed herein may be applied to the seed in the form of a suitable formulation. Suitable formulations and methods for the treatment of seed are known to the person skilled in the art and are described, for example, in the following documents: U.S. Pat. Nos. 4,272,417 A, 4,245,432 A, 4,808,430 A, 5,876,739 A, 2003/0176428 A1, WO 2002/080675 A1, WO 2002/028186 A2.

A bacterial strain disclosed herein, or a progeny, mutant, or variant thereof, a fermentate produced from a strain disclosed herein progeny, mutant, or variant thereof, and/or a depsipeptide disclosed herein can be converted into customary seed dressing formulations, such as solutions, emulsions, suspensions, powders, foams, slurries or other coating materials for seed, and also ULV formulations. These formulations are prepared in a known manner by mixing A bacterial strain disclosed herein, or a progeny, mutant, or variant thereof, a fermentate produced from a strain disclosed herein progeny, mutant, or variant thereof, and/or a depsipeptide disclosed herein with customary additives, such as, for example, customary extenders and also solvents or diluents, colorants, wetting agents, dispersants, emulsifiers, defoamers, preservatives, secondary thickeners, adhesives, gibberellins and water as well.

In another embodiment, suitable colorants that may be present in the seed dressing formulations include all colorants customary for such purposes. Use may be made both of pigments, of sparing solubility in water, and of dyes, which are soluble in water. Examples that may be mentioned include the colorants known under the designations Rhodamine B, C.I. Pigment Red 112, and C.I. Solvent Red 1.

In another embodiment, suitable wetting agents that may be present in the seed dressing formulations include all substances that promote wetting and are customary in the formulation of active agrochemical substances. With preference it is possible to use alkylnaphthalene-sulphonates, such as diisopropyl- or diisobutylnaphthalene-sulphonates.

In still another embodiment, suitable dispersants and/or emulsifiers that may be present in the seed dressing formulations include all nonionic, anionic, and cationic dispersants that are customary in the formulation of active agrochemical substances. In one embodiment, nonionic or anionic dispersants or mixtures of nonionic or anionic dispersants can be used. In one embodiment, nonionic dispersants include but are not limited to ethylene oxide-propylene oxide block polymers, alkylphenol polyglycol ethers, and tristyrylphenol polyglycol ethers, and their phosphated or sulphated derivatives.

In still another embodiment, defoamers that may be present in the seed dressing formulations to be used include all foam-inhibiting compounds that are customary in the formulation of agrochemically active compounds including but not limited to silicone defoamers, magnesium stearate, silicone emulsions, long-chain alcohols, fatty acids and their salts and also organofluorine compounds and mixtures thereof.

In still another embodiment, secondary thickeners that may be present in the seed dressing formulations include all compounds which can be used for such purposes in agrochemical compositions, including but not limited to cellulose derivatives, acrylic acid derivatives, polysaccharides, such as xanthan gum or Veegum, modified clays, phyllosilicates, such as attapulgite and bentonite, and also finely divided silicic acids.

Suitable adhesives that may be present in the seed dressing formulations may include all customary binders which can be used in seed dressings. Polyvinylpyrrolidone, polyvinyl acetate, polyvinyl alcohol and tylose may be mentioned as being preferred.

In yet another embodiment, seed dressing formulations may be used directly or after dilution with water beforehand to treat seed of any of a very wide variety of types. The seed dressing formulations or their dilute preparations may also be used to dress seed of transgenic plants. In this context, synergistic effects may also arise in interaction with the substances formed by expression.

Suitable mixing equipment for treating seed with the seed dressing formulations or the preparations prepared from them by adding water includes all mixing equipment that can commonly be used for dressing. The specific procedure adopted when dressing comprises introducing the seed into a mixer, adding the particular desired amount of seed dressing formulation, either as it is or following dilution with water beforehand, and carrying out mixing until the formulation is uniformly distributed on the seed. Optionally, a drying operation follows.

In various embodiments, a bacterial strain disclosed herein, or a progeny, mutant, or variant thereof, a fermentate produced from a strain disclosed herein progeny, mutant, or variant thereof, and/or a depsipeptide formulations can be added to the plant, plant part, and/or seed at a rate of about 1×10² to 1×10¹³ colony forming units (cfu) per seed, including about 1×10³ cfu/seed, or about 1×10⁴ cfu/seed, 1×10⁵ cfu/seed, or about 1×10⁶ cfu/seed, or about 1×10⁷ cfu/seed, or about 1×10⁸ cfu/seed, or about 1×10⁹ cfu/seed, or about 1×10¹⁰ cfu/seed, or about 1×10¹¹ cfu/seed, or about 1×10¹² cfu/seed, or about 1×10¹³ cfu/seed including about 1×10³ to 1×10⁸ cfu/seed about 1×10³ to 1×10⁷ cfu/seed, about 1×10³ to 1×10⁵ cfu/seed, about 1×10³ to 1×10⁶ cfu/seed, about 1×10³ to 1×10⁴ cfu/seed, about 1×10³ to 1×10⁹ cfu/seed, about 1×10³ to 1×10¹⁰ cfu/seed, about 1×10³ to 1×10¹¹ cfu/seed, about 1×10³ to 1×10¹² cfu/seed, about 1×10³ to 1×10¹³ cfu/seed, about 1×10⁴ to 1×10⁸ cfu/seed about 1×10⁴ to 1×10⁷ cfu/seed, about 1×10⁴ to 1×10⁵ cfu/seed, about 1×10⁴ to 1×10⁶ cfu/seed, about 1×10⁴ to 1×10⁹ cfu/seed, about 1×10⁴ to 1×10¹⁰ cfu/seed, about 1×10¹¹ to 1×10⁹ cfu/seed, about 1×10⁴ to 1×10¹² cfu/seed about 1×10⁴ to 1×10¹³ cfu/seed, about 1×10⁵ to 1×10⁷ cfu/per seed, about 1×10⁵ to 1×10⁶ cfu/per seed, about 1×10⁵ to 1×10⁸ cfu/per seed, about 1×10⁵ to 1×10⁹ cfu/per seed, about 1×10⁵ to 1×10¹⁰ cfu/per seed, about 1×10⁵ to 1×10¹¹ cfu/per seed, about 1×10⁵ to 1×10¹² cfu/per seed, about 1×10⁵ to 1×10¹³ cfu/per seed, about 1×10⁶ to 1×10⁸ cfu/per seed, about 1×10⁶ to 1×10⁷ cfu/per seed, about 1×10⁶ to 1×10⁹ cfu/per seed, about 1×10⁶ to 1×10¹⁰ cfu/per seed, about 1×10⁶ to 1×10¹¹ cfu/per seed, about 1×10⁶ to 1×10¹² cfu/per seed, about 1×10⁶ to 1×10¹³ cfu/per seed, about 1×10⁷ to 1×10⁸ cfu/per seed, about 1×10⁷ to 1×10⁹ cfu/per seed, about 1×10⁷ to 1×10¹⁰ cfu/per seed, about 1×10⁷ to 1×10¹¹ cfu/per seed, about 1×10⁷ to 1×10¹² cfu/per seed, about 1×10⁷ to 1×10¹³ cfu/per seed, about 1×10⁸ to 1×10⁹ cfu/per seed, about 1×10⁸ to 1×10¹⁰ cfu/per seed, about 1×10⁸ to 1×10¹¹ cfu/per seed, about 1×10⁸ to 1×10¹² cfu/per seed, about 1×10⁸ to 1×10¹³ cfu/per seed, about 1×10⁹ to 1×10¹⁰ cfu/per seed, about 1×10⁹ to 1×10¹¹ cfu/per seed, about 1×10⁹ to 1×10¹² cfu/per seed, about 1×10⁹ to 1×10¹³ cfu/per seed, about 1×10¹⁰ to 1×10¹¹ cfu/per seed, about 1×10¹⁰ to 1×10¹² cfu/per seed, about 1×10¹⁰ to 1×10¹³ cfu/per seed, about 1×10¹¹¹ to 1×10¹² cfu/per seed, about 1×10¹¹ to 1×10¹³ cfu/per seed, and about 1×10¹² to 1×10¹³ cfu/per seed. As used herein, the term “colony forming unit” or “cfu” is a unit capable of growing and producing a colony of a microbial strain in favorable conditions.

In one embodiment, a bacterial strain disclosed herein, or a progeny, mutant, or variant thereof, a fermentate produced from a strain disclosed herein progeny, mutant, or variant thereof, and/or a depsipeptide may be formulated as a liquid seed treatment. A seed treatment may comprise at least one a bacterial strain disclosed herein, or a progeny, mutant, or variant thereof, a fermentate produced from a strain disclosed herein progeny, mutant, or variant thereof, and/or a depsipeptide composition. The seeds are substantially uniformly coated with one or more layers of a bacterial strain disclosed herein, or a progeny, mutant, or variant thereof, a fermentate produced from a strain disclosed herein progeny, mutant, or variant thereof, and/or a depsipeptide, using conventional methods of mixing, spraying or a combination thereof. Application is done using equipment that accurately, safely, and efficiently applies seed treatment products to seeds. Such equipment uses various types of coating technology such as rotary coaters, drum coaters, fluidized bed techniques, spouted beds, rotary mists or a combination thereof.

In one embodiment, the application is done via either a spinning “atomizer” disk or a spray nozzle that evenly distributes the seed treatment onto the seed as it moves through the spray pattern. In yet another embodiment, the seed is then mixed or tumbled for an additional period of time to achieve additional treatment distribution and drying. The seeds may be primed or unprimed before coating with a composition disclosed herein to increase the uniformity of germination and emergence. In an alternative embodiment, a dry powder composition can be metered onto the moving seed.

In still another embodiment, the seeds may be coated via a continuous or batch coating process. In a continuous coating process, continuous flow equipment simultaneously meters both the seed flow and the seed treatment products. A slide gate, cone and orifice, seed wheel, or weight device (belt or diverter) regulates seed flow. Once the seed flow rate through treating equipment is determined, the flow rate of the seed treatment is calibrated to the seed flow rate in order to deliver the desired dose to the seed as it flows through the seed treating equipment. Additionally, a computer system may monitor the seed input to the coating machine, thereby maintaining a constant flow of the appropriate amount of seed.

In a batch coating process, batch treating equipment weighs out a prescribed amount of seed and places the seed into a closed treating chamber or bowl where the corresponding of seed treatment is then applied. The seed and seed treatment are then mixed to achieve a substantially uniform coating on each seed. This batch is then dumped out of the treating chamber in preparation for the treatment of the next batch. With computer control systems, this batch process is automated enabling it to continuously repeat the batch treating process.

A variety of additives can be added to the seed treatments. Binders can be added and include those composed preferably of an adhesive polymer that can be natural or synthetic without phytotoxic effect on the seed to be coated. A variety of colorants may be employed, including organic chromophores classified as nitroso, nitro, azo, including monoazo, bisazo, and polyazo, diphenylmethane, triarylmethane, xanthene, methane, acridine, thiazole, thiazine, indamine, indophenol, azine, oxazine, anthraquinone, and phthalocyanine. Other additives that can be added include trace nutrients such as salts of iron, manganese, boron, copper, cobalt, molybdenum, and zinc. A polymer or other dust control agent can be applied to retain the treatment on the seed surface.

Other conventional seed treatment additives include, but are not limited to, coating agents, wetting agents, buffering agents, and polysaccharides. At least one agriculturally acceptable carrier can be added to the seed treatment formulation such as water, solids or dry powders. The dry powders can be derived from a variety of materials such as wood barks, calcium carbonate, gypsum, vermiculite, talc, humus, activated charcoal, and various phosphorous compounds.

In one embodiment, the seed coating can comprise of at least one filler, which is an organic or inorganic, natural or synthetic component with which a bacterial strain disclosed herein, or a progeny, mutant, or variant thereof, a fermentate produced from a strain disclosed herein progeny, mutant, or variant thereof, and/or a depsipeptide described herein is combined to facilitate its application onto the seed. In one embodiment, the filler is an inert solid such as clays, natural or synthetic silicates, silica, resins, waxes, solid fertilizers (for example ammonium salts), natural soil minerals, such as kaolins, clays, talc, lime, quartz, attapulgite, montmorillonite, bentonite, or diatomaceous earths, or synthetic minerals, such as silica, alumina, or silicates, in particular aluminum or magnesium silicates.

In one embodiment, a bacterial strain disclosed herein, or a progeny, mutant, or variant thereof, a fermentate produced from a strain disclosed herein progeny, mutant, or variant thereof, and/or a depsipeptide disclosed herein may be formulated by encapsulation technology to improve fungal spore stability and as a way to protect the fungal spores from seed applied fungicides. In one embodiment the encapsulation technology may comprise a bead polymer for timed release of fungal spores over time. In one embodiment, the encapsulation technology may comprise a zeolite material. In one embodiment, an encapsulated bacterial strain disclosed herein, or a progeny, mutant, or variant thereof, fermentate produced from a strain disclosed herein progeny, mutant, or variant thereof, and/or depsipeptide may be applied in a separate application of beads in-furrow to the seeds. In another embodiment, the encapsulated bacterial strain disclosed herein, or a progeny, mutant, or variant thereof, fermentate produced from a strain disclosed herein progeny, mutant, or variant thereof, and/or depsipeptide may be co-applied along with seeds simultaneously.

Insect resistance management (IRM) is the term used to describe practices aimed at reducing the potential for insect pests to become resistant to an insect management tactic. IRM maintenance of Bt (Bacillus thuringiensis) derived pesticidal proteins, other pesticidal proteins, a chemical, a biological agent, or other biologicals, is of great importance because of the threat insect resistance poses to the future use of pesticidal plant-incorporated protectants and insecticidal trait technology as a whole. Specific IRM strategies, such as the refuge strategy, mitigate insect resistance to specific insecticidal proteins produced in corn, soybean, cotton, and other crops. However, such strategies result in portions of crops being left susceptible to one or more pests in order to ensure that non-resistant insects develop and become available to mate with any resistant pests produced in protected crops. Accordingly, from a farmer/producer's perspective, it is highly desirable to have as small a refuge as possible and yet still manage insect resistance, in order that the greatest yield be obtained while still maintaining the efficacy of the pest control method used, whether Bt, a different pesticidal protein, chemical, biological agent or other biologicals, some other method, or combinations thereof.

Another strategy to reduce the need for refuge is the pyramiding of traits with different modes of action against a target insect pest. For example, Bt toxins that have different modes of action pyramided in one transgenic plant are able to have reduced refuge requirements due to reduced resistance risk. Different modes of action in a pyramid combination also extend the durability of each trait, as resistance is slower to develop to each trait.

Currently, the size, placement, and management of the refuge are often considered critical to the success of refuge strategies to mitigate insect resistance to the Bt/pesticidal trait produced in corn, cotton, soybean, and other crops. Because of the decrease in yield in refuge planting areas, some farmers choose to eschew the refuge requirements, and others do not follow the size and/or placement requirements. These issues result in either no refuge or a less effective refuge, and a corresponding risk of the increase in the development of resistance pests.

Accordingly, there remains a need for methods for managing pest resistance in a plot of pest resistant crop plants. It would be useful to provide an improved method for the protection of plants, especially corn or other crop plants, from feeding damage by pests. It would be particularly useful if such a method would reduce the required application rate of conventional chemical pesticides, and also if it would limit the number of separate field operations that were required for crop planting and cultivation. In addition, it would be useful to have a method of deploying a biocontrol agent that increases the durability of an insecticidal trait or increases the efficacy of many resistance management strategies.

One embodiment relates to a method of reducing or preventing the resistance of pests to a plant pesticidal composition comprising providing a plant protection composition, such as a Bt pesticidal protein, a transgenic pesticidal protein, other pesticidal proteins, chemical pesticides, or pesticidal biological entomopathogens, to a plant and/or plant part or a planted area or insecticidal trait and providing a bacterial strain disclosed herein, or a progeny, mutant, or variant thereof, a fermentate produced from a strain disclosed herein progeny, mutant, or variant thereof, and/or a depsipeptide described herein to the plant and/or plant part or planted area. Another embodiment relates to a method of reducing or preventing the resistance to a plant insecticidal trait comprising providing or contacting a plant with a bacterial strain disclosed herein, or a progeny, mutant, or variant thereof, a fermentate produced from a strain disclosed herein progeny, mutant, or variant thereof, and/or a depsipeptide described herein.

A further embodiment relates to a method of increasing the durability of plant pest compositions comprising providing a plant protection composition to a plant or planted area, and providing a bacterial strain disclosed herein, or a progeny, mutant, or variant thereof, a fermentate produced from a strain disclosed herein progeny, mutant, or variant thereof, and/or a depsipeptide described herein to the plant or planted area, wherein the bacterial strain disclosed herein, or a progeny, mutant, or variant thereof, fermentate produced from a strain disclosed herein progeny, mutant, or variant thereof, and/or depsipeptide described herein have a different mode of action than the plant protection composition.

In a still further embodiment, the refuge required may be reduced or eliminated by the presence of a bacterial strain disclosed herein, or a progeny, mutant, or variant thereof, a fermentate produced from a strain disclosed herein progeny, mutant, or variant thereof, and/or a depsipeptide described herein applied to the non-refuge plants. In another embodiment, the refuge may include a bacterial strain disclosed herein, or a progeny, mutant, or variant thereof, a fermentate produced from a strain disclosed herein progeny, mutant, or variant thereof, and/or a depsipeptide described herein as a spray, bait, or as a different mode of action.

In one embodiment, a composition comprises a bacterial strain disclosed herein, or a progeny, mutant, or variant thereof, a fermentate produced from a strain disclosed herein progeny, mutant, or variant thereof, and/or a depsipeptide disclosed herein and a non-Bt insecticidal trait increases resistance to a pathogen, pest, or insect. In another embodiment, the non-Bt insecticidal trait comprises a plant-derived insecticidal protein, a bacterial/archeal-derived insecticidal protein not from a Bt (such as a Pseudomonas insecticidal protein), an animal-derived insecticidal protein, or a silencing element. In another embodiment, a composition comprising a bacterial strain disclosed herein, or a progeny, mutant, or variant thereof, a fermentate produced from a strain disclosed herein progeny, mutant, or variant thereof, and/or a depsipeptide disclosed herein and a non-Bt insecticidal trait increases durability of the non-Bt insecticidal trait. In another embodiment, the non-Bt insecticidal trait comprises a PIP-72 polypeptide of PCT Serial Number PCT/US14/55128. In another embodiment, the non-Bt insecticidal trait comprises a polynucleotide silencing elements targeting RyanR (DvSSJ) (US Patent Application publication 2014/0275208). In another embodiment, the non-Bt insecticidal trait comprises a polynucleotide silencing elements targeting RyanR (DvSSJ) (US Patent Application publication 2014/0275208, herein incorporated by reference in its entirety) and a PIP-72 polypeptide of PCT Serial Number PCT/US14/55128, herein incorporated by reference in its entirety.

In another embodiment, a composition comprising a bacterial strain disclosed herein, or a progeny, mutant, or variant thereof, a fermentate produced from a strain disclosed herein progeny, mutant, or variant thereof, and/or a depsipeptide disclosed herein and a fungal entomopathogen disclosed in U.S. Pat. No. 9,993,006, herein incorporated by reference in its entirety.

In some embodiments, a composition comprises a bacterial strain disclosed herein, or a progeny, mutant, or variant thereof, a fermentate produced from a strain disclosed herein progeny, mutant, or variant thereof, and/or a depsipeptide disclosed herein and a Bt insecticidal trait that increases resistance to a pathogen, pest, or insect. A Bt insecticidal trait may have activity to Coleopteran, Lepidopteran, or Hemipteran plant pests. The compositions disclosed herein may provide to a plant or plant part additive or synergistic resistance to a pathogen, pest, or insect plant in combination with a Bt insecticidal trait. In one embodiment, a composition comprises a bacterial strain disclosed herein, or a progeny, mutant, or variant thereof, a fermentate produced from a strain disclosed herein progeny, mutant, or variant thereof, and/or a depsipeptide disclosed herein and a Bt insecticidal trait, wherein the Bt insecticidal trait comprises a Cry3B toxin disclosed in U.S. Pat. Nos. 8,101,826, 6,551,962, 6,586,365, 6,593,273, and PCT Publication WO 2000/011185, a mCry3B toxin disclosed in U.S. Pat. Nos. 8,269,069, and 8,513,492, a mCry3A toxin disclosed in U.S. Pat. Nos. 8,269,069, 7,276,583 and 8,759,620, or a Cry34/35 toxin disclosed in U.S. Pat. Nos. 7,309,785, 7,524,810, 7,985,893, 7,939,651 and 6,548,291, and transgenic events containing these Bt insecticidal toxins and other Coleopteran active Bt insecticidal traits for example, event MON863 disclosed in U.S. Pat. No. 7,705,216, event MIR604 disclosed in U.S. Pat. No. 8,884,102, event 5307 disclosed in U.S. Pat. No. 9,133,474, event DAS-59122 disclosed in U.S. Pat. No. 7,875,429, event DP-4114 disclosed in U.S. Pat. No. 8,575,434, event MON 87411 disclosed in US Published Patent Application Number 2013/0340111, and event MON88017 disclosed in U.S. Pat. No. 8,686,230 all of which are incorporated herein by reference. All publications, patents and patent applications mentioned in the specification indicate the level of those skilled in the art to which this disclosure pertains. All publications, patents and patent applications are incorporated by reference to the same extent as if each individual publication, patent or patent application was specifically and individually indicated to be incorporated by reference.

A bacterial strain disclosed herein, or a progeny, mutant, or variant thereof, a fermentate produced from a strain disclosed herein progeny, mutant, or variant thereof, and/or a depsipeptide compositions and methods will be further understood by reference to the following non-limiting examples. The following Examples are provided for illustrative purposes only. The Examples are included solely to aid in a more complete understanding of the described embodiments. The Examples do not limit the scope of the embodiments described or claimed.

### EXAMPLE 1

### Identification of Pseudomonas chlororaphis Strain SS532D1 (NRRL Deposit No. B-67638) with Toxicity against Fall Armyworm

Insecticidal activity against Fall Armyworm (FAW, Spodoptera frugiperda) was observed in the fermented broth from soil sample-derived microbial strain SS532D1. It was grown in Tryptic Soy Broth (Soybean-Casein Digest Medium: Tryptone—17 g/L, Soytone—3 g/L, Glucose—2.5 g/L, Sodium Chloride—5 g/L and Dipotassium hydrogen phosphate—2.5 g/L) at 26° C. for 2 days with shaking at 210 rpm. Genomic DNA from strain SS532D1 was extracted with a Sigma Bacterial Genomic DNA Extraction Kit (Cat #NA2110-KT, Sigma-Aldrich, PO Box 14508, St. Louis, Mo. 63178) according to the manufactures' instructions. The DNA concentration was determined using a NanoDrop Spectrophotometer (Thermo Scientific) and the genomic DNA was diluted to 40 ng/ul with sterile water. A 25 ul PCR reaction was set up by combining 80 ng genomic DNA, 2 ul (5 uM) 16S ribosomal DNA primers TACCTTGTTACGACTT (SEQ ID NO: 209) and AGAGTTTGATCMTGGCTCAG (SEQ ID NO: 210), 1 ul 10 cmM dNTP, 1× Phusion HF buffer, and 1 unit of Phusion High-Fidelity DNA Polymerase (New England Biolabs). The PCR reaction was run in MJ Research PTC-200 Thermo Cycler (Bio-Rad Laboratories, Inc., California) with the following program: 96° C. 1 min; 30 cycles of 96° C. 15 seconds, 52° C. 2 minutes and 72° C. 2 minutes; 72° C. 10 minutes; and hold on 4° C. The PCR products were purified with QiaQuick DNA purification Kit (QIAGEN Inc., California). The purified PCR sample was sequenced and the resulting 16S ribosomal DNA sequence (SEQ ID NO:1) was BLAST searched against the NCBI database which indicated that SS532D1 is a Pseudomonas chlororaphis strain.

### EXAMPLE 2

### Leaf Painting Assay on FAW with SSP532D1 Culture

Strain SSP532D1 was grown in Tryptic soy broth (TSB, tryptone—17 g/L, soytone—3 g/L, glucose—2.5 g/L, sodium chloride—5 g/L and dipotassium hydrogen phosphate—2.5 g/L) at 26 C for 2 days. The culture was diluted as 1×, 1/2 and 1/4× with Milli-Q water. Twenty-five microliters of undiluted or diluted cultures was painted on each bush bean leaf disk. After being air-dried, leaf disks were placed on the top of agar (1%) in 48-well plates. Three FAW neonate larvae were placed on top of the leaf disk in each well. Results were scored after 3 days. Insects were killed on leaf disks treated with undiluted and diluted SSP532D1 culture and the leaf disks showed no obvious feeding damage. Insects on leaf disks treated with non-active cultures consumed a significant amount of the leaf disks.

### EXAMPLE 3

### Lepidoptera Diet-Based Feeding Assays

Lepidopteran feeding assays (including FAW) were conducted on an artificial diet containing fermented broth in a 96 well plate set up. The broth was incorporated with multi-species Lepidopteran artificial diet (Southland Products Inc., Arkansas) in a ratio of 25 μl test sample and 35 μl of diet. Two to five neonate larvae were placed in each well to feed for 4 days. Results were expressed as positive for larvae reactions such as stunting and/or mortality. Results were expressed as negative if the larvae were similar to the negative control that is fed diet to which the above control broth only has been applied.

### Coleoptera Diet Based Feeding Assays

Western Corn Rootworm (WCRW, Diabrotica virgifera virgifera) bioassays were conducted in a 96 well plate format, mixing 10 μl of test sample with 50 μl molten modified Southern Corn Rootworm diet (Frontier Agricultural Sciences, diet F9800B). WCRW neonates were placed into each well before heat sealed with a Mylar film. Holes are punched in the film to allow air exchange. The assay was run four days at 25° C. in the dark. The plates were then scored for insect mortality and stunting of insect growth. The scores were noted as dead, severely stunted (little or no growth but alive), stunted (growth but not equivalent to controls) or no activity.

### Hemiptera Diet Based Feeding Assays

Southern green stinkbug (Nezara viridula) bioassays were conducted using 40 ul of fermented medium was mixed with 360 ul of Lygus diet (Bio-Sery F9644B) in Parafilm® packets. 10 to 15 newly molted second instar nymphs were placed in polystyrene Petri dishes (100 mm×20 mm) lined with moist Whatman® filter paper (100 mm diameter). Included in the dish was a water source. The bioassay was incubated at 25° C. in the dark for three days and the then the diet/sample packet was replaced. The bioassay was scored for mortality and stunting after a total of 6 days.

### EXAMPLE 4

### Characterization of Insecticidal Activity in Fermented Broth from Strain SS532D1

Fermented broth from strain SS532D1 grown for 2 days in Tryptic soy broth was tested for activity against several other insect species. Broad spectrum activity was observed. Undiluted fermented broth mixed with artificial diet, as described in Example 3, caused mortality in the following species: European corn borer (ECB, Ostrinia nubilalis), fall armyworm (FAW, Spodoptera frugiperda), soybean looper (SBL, Pseudoplusia includens), velvet bean caterpillar (VBC, Anticarsia gemmatalis), diamondback moth (DBM, Plutella xylostella), Western corn rootworm (WCRW, Diabrotica virgifera) and black bean aphid (Aphis fabae). Severe stunting was observed against Southern corn rootworm (Diabrotica undecimpunctata howardi) and corn earworm (Hehcoverpa zea). No activity was found against Southern green stinkbug (Nezara viridula) as tested.

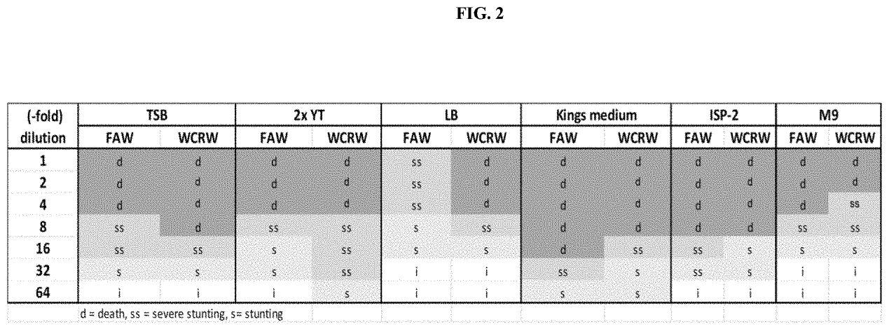

To maximize insecticidal activity in the fermented broth several growth media were tested against FAW and WCRW, specifically Tryptic soy broth (TSB, tryptone—17 g/L, soytone—3 g/L, glucose—2.5 g/L, sodium chloride—5 g/L and dipotassium hydrogen phosphate—2.5 g/L), 2×YT medium (Bacto Tryptone—16 g/L, Bacto Yeast Extract—10 g/L, sodium chloride—5 g/L), Luria Broth (LB, Tryptone Peptone—10 g/L, Yeast Extract 5-g/L, sodium chloride—10 g/L), King's medium (Peptone—20 g/L, Glycerol—15 ml/L, K₂HPO₄—1.5 g/L, MgSO₄*7H₂0—1.5 g/L) ISP-2 medium (Yeast Extract—4.0 g/L, Malt Extract—10.0 g/L, Dextrose—4.0 g/L) and M9 minimal medium (Na₂HPO₄×7H₂O—12.8 g/L, KH₂PO₄—3 g/L, NaCl—0.5 g/L, NH₄Cl—1 g/L, CaCl₂—1.1 mg/L, MgSO₄—240.7 mg/L, glucose—4 g/L). Strain SS532D1 was grown at 26° C. at 210 rpm. After 2 days, growth medium was harvested by centrifugation and bioassays were performed on FAW and WCRW. All spent media showed insecticidal activity on both insects, with King's medium showing the highest potency. The results are summarized in FIG. 2. Insecticidal activity against FAW was highest at 1 to 2 days of growth in King's medium and declined after 3 and 6 days of fermentation.

### EXAMPLE 5

### Lepidoptera Small-Scale, Plant-Based Assays

Two types of broth produced after fermentation of strain SS532D1, herein referred to as Broth A and Broth B. Broth A was produced in King's Media (King's) and Broth B was produced in M9 Minimal Media (M9). Both media contained the same depsipeptide after fermentation. The only difference from Broth A to King's Media and Broth B was produced in M9 Minimal Media being that the Broth A and B were used to grow the bacterium, Pseudomonas chlororaphis strain SS532D1.

Broths A (King's Media) and B (M9 Minimal Media) were tested against DBM and FAW in small-scale foliar assays. Test units were comprised of 20 cm³ pots with 20 g of Woodstown Sandy Loam soil (Delmar, Del.) and mustard (Brassicae kaber, 3.5-4 cm in height) or corn (Zea mays, 6 cm) as host plants for DBM and FAW, respectively. Test compounds were sprayed on plants in a 120 μL volume and allowed to dry for 4 hours prior to infestation. Test units were infested with approximately 15-20 DBM and FAW neonates and kept in controlled conditions of 25° C./70% RH/16:8 h (L:D) photoperiod. After six days, assays were scored using a binary scoring system in which plant damage=0 and no plant damage=1. The results are reported as percent activity, which are the averages of the three technical replicates per treatment in the assay. Treatments included undiluted Broths A and B, unspent King's and M9 Minimal media (fresh media without bacterial growth), and untreated controls. Broth A controlled both DBM and FAW (Table 2). Broth B was effective against DBM, but only partially against FAW (33.3%). No activity was observed in untreated or unspent media controls.

TABLE 2
Activity of Broth A and Broth B on diamondback moth
and fall armyworm in small-scale foliar assays.

| Treatment | DBM | FAW |
|---|---|---|
| Untreated Control | 0 | 0 |
| King's | 0 | 0 |
| M9 | 0 | 0 |
| Broth A | 100 | 100 |
| Broth B | 100 | 33.3 |

### EXAMPLE 6

### Lepidoptera Medium-Sized, Plant-Based Assays

Broths A and B were tested against four insect species in medium insect test units; DBM, FAW, beet armyworm (Spodoptera exigua; BAW), and corn earworm (Helicoverpa zea; CEW). Test units were comprised of 70 cm³ bases with 35 g of Woodstown Sandy Loam soil (Delmar, Del.) and mustard (Brassicae kaber; 6.5-7 cm), corn (Zea mays; 7-8 cm) or Bush bean (Phaseolus vulgaris; 6-7cm) as host plants for DBM, FAW and BAW, and CEW, respectively. Three concentrations of Broths A and B were tested: 0.25, 0.5 and 1×, with and without the surfactant X-77. Spinetoram was included as a positive control. Untreated controls were also included. Test units were sprayed with 1.2 mL of broth, in triplicate, and subsequently infested with approximately 15-20 stage 1 BAW or FAW larvae, or 30-40 neonate DBM or CEW. Units were kept in controlled conditions of 25° C./70% RH/16:8 h (L:D) photoperiod for six days and then scored. There are two scores given for each treatment, (1) plant damage rating from 0-10, with 0 being no damage and 10 being plant was completely consumed, and (2) insect live or dead (live=F; dead=K). The scores are averages of the three technical replicates. Any treatment with a plant damage score of 3 or less and an insect rating of K was considered to be active.

Tables 3 and 4 summarize the data obtained for Broths A and B without and with the surfactant X-77, respectively. For the assay without the surfactant, Broth A was active against all four species at all three concentrations except for 0.5× for CEW (Table 3). Broth B was active against BAW at all three concentrations. Broth B was also active against FAW at 0.5 and 1×. However, Broth B showed no activity against CEW as tested. Unspent King's and M9 media were both active against DBM (data not shown). Otherwise, all other controls performed within expected parameters. Overall, the results obtained for Broth A formulated with the surfactant X-77 were similar to those obtained for the broth without the surfactant. However, the efficacy of Broth B was reduced when formulated with X-77, in particular, against FAW (Table 4).

TABLE 3
Activity of Broth A and Broth B against beet
armyworm, fall armyworm and corn earworm.

| | dilution | | | |
|---|---|---|---|---|
| | or ppm | BAW | FAW | CEW |
| Untreated Control | None | 10 F | 10 F | 10 F |
| King's | 0 | 10 F | 10 F | 9 F |
| M9 | 0 | 10 F | 10 F | 8 F |
| Broth A | 1X | 1 K | 1 K | 2 K |
| | 0.5X | 1 K | 2 K | 4 F |
| | 0.25X | 1 K | 3 K | 3 K |
| Broth B | 1X | 1 K | 3 K | 4 K |
| | 0.5X | 1 K | 3 K | 8 F |
| | 0.25X | 1 K | 9 F | 10 F |
| Spinetoram | 2 | 0 K | 0 K | 0 K |
| | 0.2 | 4 F | 1 K | 1 K |
| | 0.1 | 7 F | 5 F | 1 K |
| | 0.03 | | | |
| | 0.01 | | | |
| | 0.003 | | | |

TABLE 4
Activity of Broth A and Broth B formulated with the surfactant
X-77 against beet armyworm, fall armyworm and corn earworm.

| | dilution | | | |
|---|---|---|---|---|
| | or ppm | BAW | FAW | CEW |
| Untreated Control | None | 10 F | 10 F | 10 F |
| King's | 0 | 10 F | 10 F | 4 K |
| M9 | 0 | 10 F | 10 F | 7 F |
| Broth A | 1X | 1 K | 2 K | 2 K |
| | 0.5X | 1 K | 3 K | 2 F |
| | 0.25X | 1 K | 3 K | 3 F |
| Broth B | 1X | 1 K | 4 F | 7 F |
| | 0.5X | 1 K | 7 F | 8 F |
| | 0.25X | 1 K | 10 F | 9 F |
| Spinetoram | 2 | 4 F | 0 K | 0 K |
| | 0.2 | 4 F | 1 K | 1 K |
| | 0.1 | 7 F | 5 F | 1 K |
| | 0.03 | | | |
| | 0.01 | | | |
| | 0.003 | | | |

### EXAMPLE 7

### Isolation and Identification of the Active Insecticidal Component

Growth medium from M9 minimal media inoculated with strain SS532D1 was harvested by centrifugation. 100% Acetonitrile was added to the harvested medium to a final concentration of 20% and minor precipitation was removed by centrifugation.

The active insecticidal component was isolated using a Chemstation® 1200 series HPLC system (Agilent®) with a C-18 reverse phase column (Phenomenex). The HPLC was prepared with trifluoroacetic acid diluted to 0.1% in Milli Q® water (Mobile Phase A) and 100% Acetonitrile with 0.1% trifluoroacetic acid included (Mobile Phase B). The C-18 reverse phase column was equilibrated with 20% Mobile Phase B. Adjusted growth medium was injected onto the column and the following method was performed: 2 column volume (CV) hold at 20% Mobile Phase B, 12 CV linear gradient from 20% Mobile Phase B to 80% Mobile Phase B, 100% Mobile Phase B hold for 2 CV, 20% Mobile Phase B hold for 5 CV. UV absorbance was monitored at 220 nm and elution fractions were collected during the linear gradient. Elution fractions were lyophilized and reconstituted in water for testing on Lepidoptera feeding assay in artificial diet as described in Example 3 to identify the fractions containing the pure toxin.

### EXAMPLE 8

### Structural Determination by Mass Spectrometry

The mass spectrometry data was obtained on a Thermo Q Exactive Orbitrap® mass spectrometer (Thermo Fisher Scientific®) interfaced with an Eksigent NanoLC Ultra 1-D Plus® nano-lc system and a nanolc-as2 auto sampler (AB Sciex).

The mass resolution was set at 70k for MS¹ scans and 17500 for MS² scans. For MS² scans the normalized collision energy (NCE) was set at 15 eV, 27 eV or 35 eV. The MS³ spectra were obtained by fragmenting the peptides with in-source fragmentation at 75 eV followed by MS² scans of the in-source fragment ions at 27 eV. All MS² scans were obtained in data dependent acquisition (DDA) mode. External mass calibration was carried out before the sample analysis.

The peptides were either separated on a nano-lc column self-packed with a New Objective PicoTip® nanosrapy emitter (15 cm length, 75 mm ID and 15 micron tip, New) Objective®) and 5 micron particle size reversed-phased C18 (Magic C18, Michrom Bioresources®) or directly eluted into the mass spectrometer from a C18-based trap column. The nano-LC mobile phase A was 0.1% formic acid in water and the mobile phase B was 0.1% formic acid in acetonitrile. The gradient program was 5% B for 1 min. followed by an increase of B to 90% in 29 min. and then 10 min. in 90% B with a flow rate at 300 nL/min.

LC/MS/MS methods and results (elemental composition) identification of depsipeptide structure FR901375 by database search confirmation of depsipeptide by LC-MS/MS/MS fragment ion analysis (Bogdan). Structure was confirmed by 2D NMR.

### EXAMPLE 9

### Potency of Depsipeptide Structure FR901375 against FAW

Strain SS531D2 was grown in Kings Medium for 2 days. The fermentate was harvested by centrifugation and filter sterilized. The presence and concentration of the depsipeptide was determined by LC-MS/MS using multiple reaction monitoring (MRM) as described. The concentration in the fermentate was 55 ug/ml. Serial dilutions were prepared and bioactivity against WCRW and several lepidopteran species was assessed in artificial diet feeding assays as described in Example 3. The potency of the depsipeptide is summarized in Table 5.

TABLE 5
The potency of depsipeptide on insects

| Insect | minimal dose to achieve 100% mortality | |
|---|---|---|
| Western Corn Rootworm | 23 | ppm |
| Fall Armyworm | 2 | pmm |
| Corn Earworm | 11 | ppm |
| European Corn Borer | 2 | ppm |
| Soybean Looper | 1 | ppm |

### EXAMPLE 10

### Identification of other Depsipeptide Producing Strains

In the screening of more bacterial collections, multiple strains showed broad spectrum insecticidal activity. The species of those active strains were identified by 16S sequences as described in Example 1. They include Pseudomonas chlororaphis strain SSP459B9-3, Burkholderia rinojensis strain JH58178-1, and Chromobacterium haemolyticum strains JH97285-1, JH91791-1, PMC3591F10-1, PMCJ4191H4-1, and PMCJ4232B7-1 (Table 6). All these strains produce depsipeptides which contribute to their insecticidal activities. Further analysis, as set forth in Example 8, found a second depsipeptide structure, FK228, was produced in some strains

TABLE 6
Depsipeptide producing strains and their species, active
spectrum, NRRL # and 16S rDNA sequences

| | | Insecticidal | NRRL Deposit | 16S rDNA |
|---|---|---|---|---|
| Strain | Species | Activity | Number | SEQ ID NO: |
| SSP459B9-3 | Pseudomonas | SBL, FAW, CEW, | NRRL B-67639 | 2 |
| | chlororaphis | VBC, ECB, WCRW | | |
| JH59178-1 | Burkholderia | SBL, FAW, CEW, | NRRL B-67640 | 3 |
| | rinojensis | VBC, ECB, WCRW | | |
| JH97285-1 | Chromobacterium | SBL, FAW, CEW, | NRRL B-67641 | 4 |
| | haemolyticum | VBC, ECB, WCRW | | |
| JH91791-1 | Chromobacterium | SBL, FAW, CEW, | NRRL B-67642 | 5 |
| | haemolyticum | VBC, ECB, WCRW | | |
| PMC3591F10-1 | Chromobacterium | SBL, FAW, CEW, | NRRL B-67643 | 6 |
| | haemolyticum | VBC, ECB, WCRW | | |
| PMCJ4191H4-1 | Chromobacterium | SBL, FAW, CEW, | NRRL B-67644 | 7 |
| | haemolyticum | VBC, ECB, WCRW | | |
| PMCJ4232B7-1 | Chromobacterium | SBL, FAW, CEW, | NRRL B-67645 | 8 |
| | haemolyticum | VBC, ECB, WCRW | | |

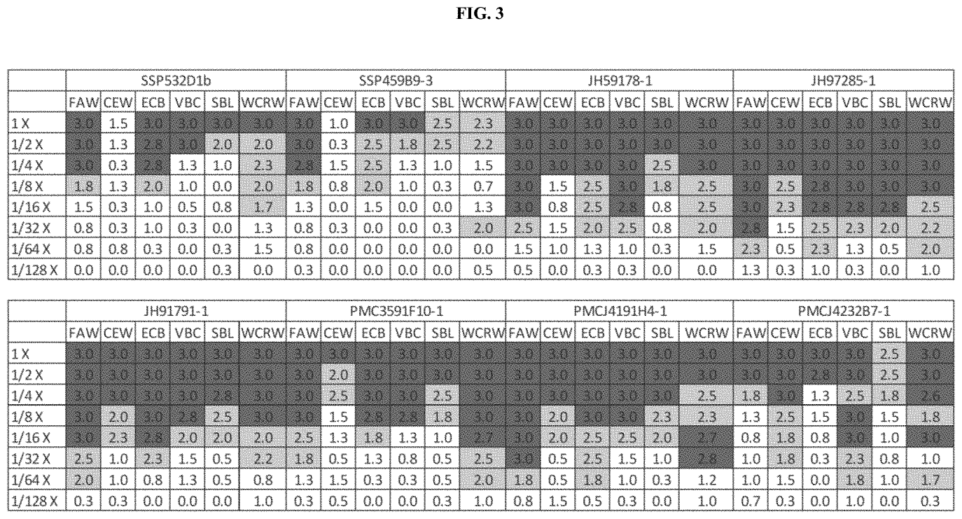

These strains were cultured as described in Example 1 and the cultures were diluted to 2, 4, 8, 16, 32, 64, and 128 folds, and tested on multiple insects. The results were summarized in FIG. 3.

### EXAMPLE 11

### Identification of Depsipeptide Biosynthesis Pathway Genes

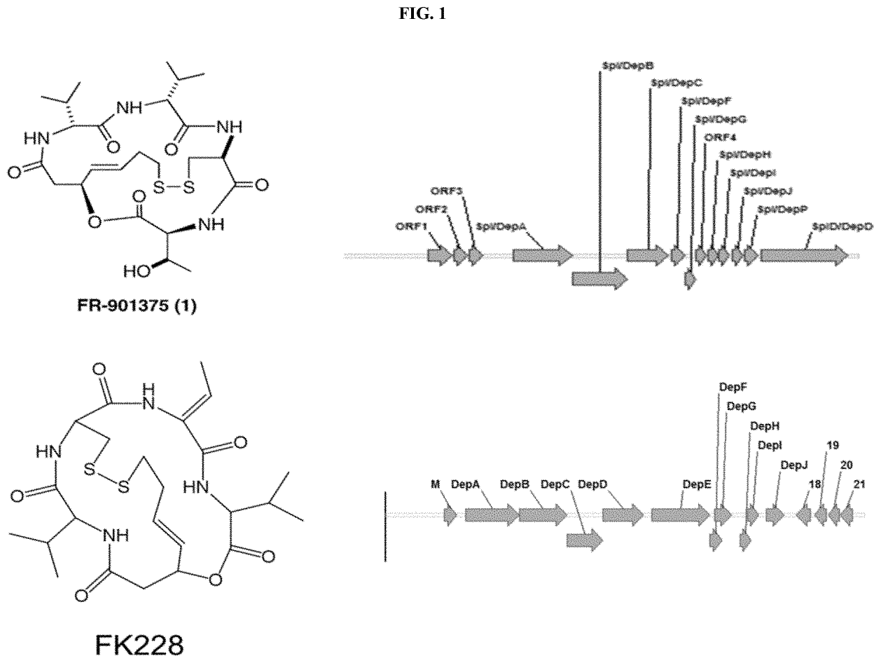

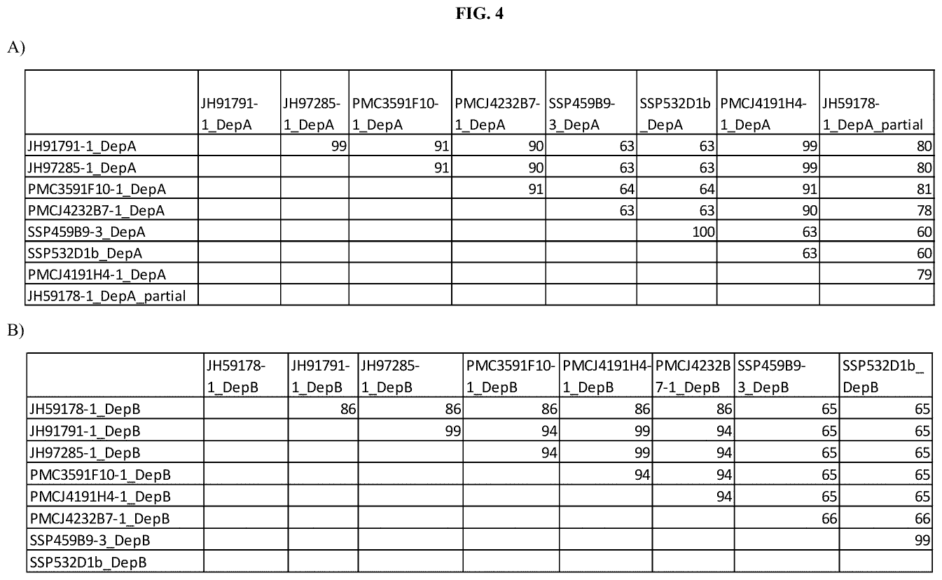

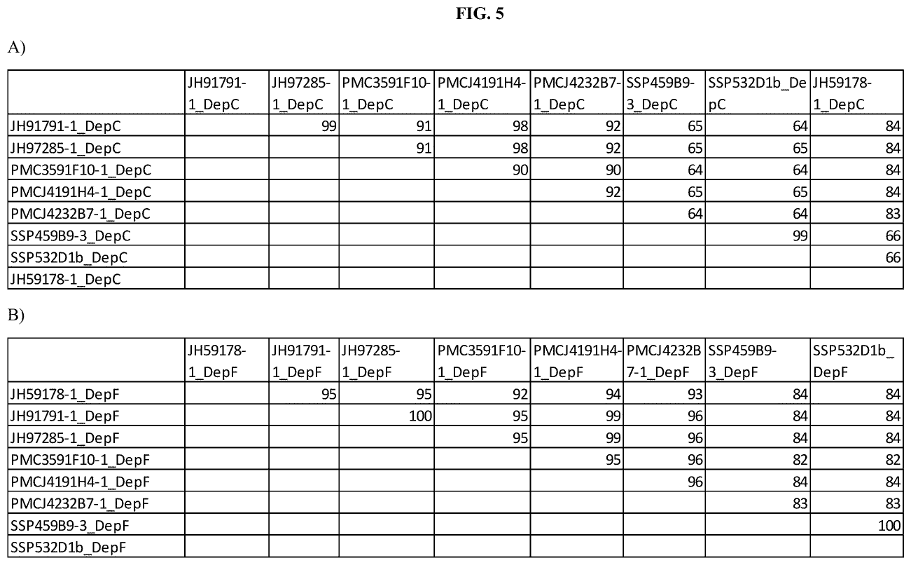

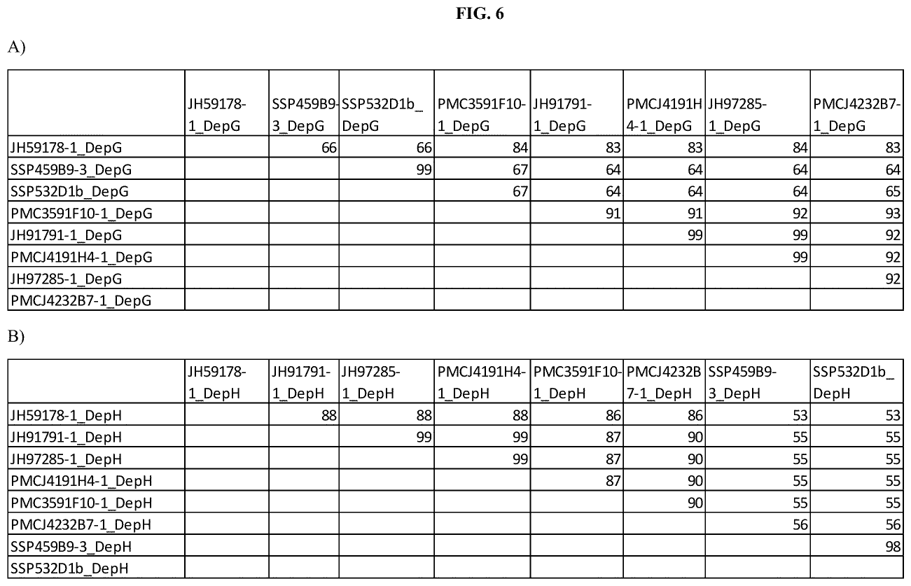

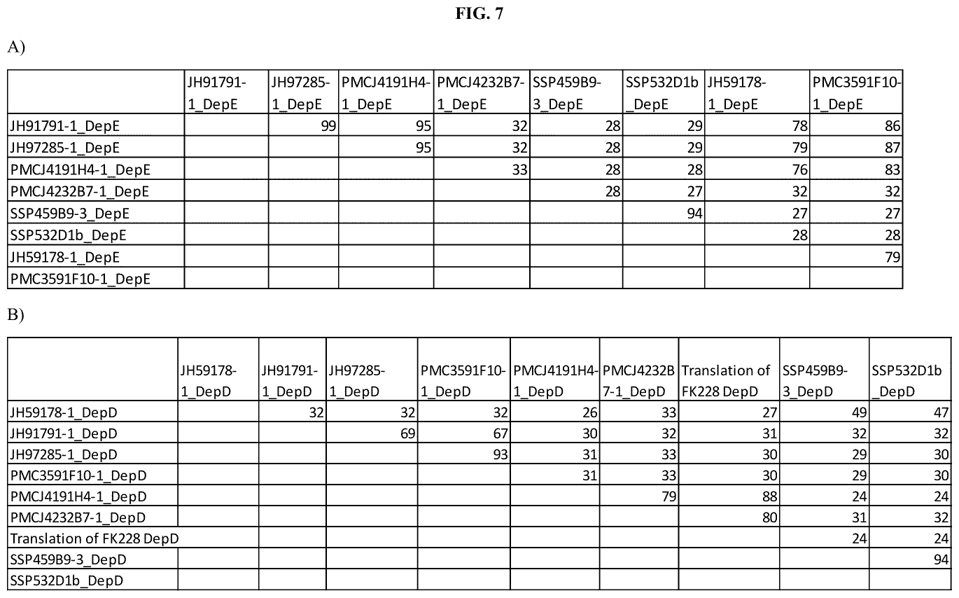

Further analysis of the biosynthesis pathway of these strains revealed that there are two different pathways which produce the two types of depsipeptides FR901375 and FK228. Pseudomonas chlororaphis strain SSP532D1b and SSP459B9-3 produce depsipeptide FR-901375; Burkholderia rinojensis strain JH58178-1, and Chromobacterium haemolyticum strain JH97285-1, JH91791-1, PMC3591F10-1, PMCJ4191H4-1, and PMCJ4232B7-1 produce depsipeptide Fk228. Multiple genes, including but not limited to DepA, DepB, DepC, DepD, DepE, DepF, DepG, DepH, DepI, DepJ, DepK, DepL, and DepM, are involved for the depsipeptide biosynthesis (FIG. 1). The percentage of sequence alignment of some of these genes are illustrated in FIGS. 4-7.

TABLE 7
Depsipeptide producing strains and depsipeptide types

| | | | Depsipeptide |
|---|---|---|---|
| Strain ID | Species | NRRL# | type |
| SSP532D1b | Pseudomonas chlororaphis | NRRL B-67638 | FR901375 |
| SSP459B9-3 | Pseudomonas chlororaphis | NRRL B-67639 | FR901375 |
| JH59178-1 | Burkholderia rinojensis | NRRL B-67640 | FK228 |
| JH97285-1 | Chromohacterium haemolyticum | NRRL B-67641 | FK228 |
| JH91791-1 | Chromohacterium haemolyticum | NRRL B-67642 | FK228 |
| PMC3591F10-1 | Chromohacterium haemolyticum | NRRL B-67643 | FK228 |
| PMCJ4191H4-1 | Chromohacterium haemolyticum | NRRL B-67644 | FK228 |
| PMCJ4232B7-1 | Chromohacterium haemolyticum | NRRL B-67645 | FK228 |

What is claimed is:

1. A composition comprising an agriculturally acceptable carrier, a plant or a plant part, and a bacterial strain having a 16S rDNA sequence comprising at least 95% sequence identity to any one of SEQ ID NOs: 1-9, wherein the bacterial strain further comprises a DepA gene, a DepB gene, a DepC gene, a DepF gene, a DepG gene, DepH gene, a DepE gene, or a DepD gene, and wherein the bacterial strain has insecticidal activity.

2. The composition of claim 1, wherein the bacterial strain is a Pseudomonas chlororaphis, a Burkholderia rinojensis, or a Chromobacterium haemolyticum.

3. The composition of claim 1, wherein the bacterial strain is a Pseudomonas chlororaphis strain SS532D1 (NRRL Deposit No. B-67638), a Pseudomonas chlororaphis strain SSP459B9-3 (NRRL Deposit No. B-67639), a Burkholderia rinojensis strain JH59178-1 (NRRL Deposit No. B-67640), a Chromobacterium haemolyticum strain JH91791-1 (NRRL Deposit No. B-67642), a Chromobacterium haemolyticum strain PMCJ4191H4-1 (NRRL Deposit No. B-67644), a Chromobacterium haemolyticum strain JH97285-1 (NRRL Deposit No. B-67641), or a Chromobacterium haemolyticum strain PMC3591F10-1 (NRRL Deposit No. B-67643), or a progeny, mutant, or variant thereof.

4. The composition of claim 1, wherein the bacterial strain produces a depsipeptide as a metabolite.

5. The composition of claim 4, wherein the depsipeptide has the structure of FR901375, FK228, or a derivative or variant thereof.

6. The composition of claim 1, wherein the DepA gene comprises a nucleic acid sequence encoding a polypeptide, wherein the polypeptide has at least 90% sequence identity to any one of SEQ ID NOs: 109, 121, 133, 145, 158, 171, 184, or 197.

7. The composition of claim 6, wherein the nucleic acid sequence comprises at least 90% sequence identity to any one of SEQ ID NOs: 9, 21, 33, 45, 58, 71, 84, or 97.

8. The composition of claim 1, wherein the DepB gene comprises a nucleic acid sequence encoding a polypeptide, wherein the polypeptide has at least 90% sequence identity to any one of SEQ ID NOs: 110, 122, 134, 146, 159, 172, 185, or 198.

9. The composition of claim 8, wherein the nucleic acid sequence comprises at least 90% sequence identity to any one of SEQ ID NOs: 10, 22, 34, 46, 59, 72, 85, or 98.

10. The composition of claim 1, wherein the DepC gene comprises a nucleic acid sequence encoding a polypeptide, wherein the polypeptide has at least 90% sequence identity to any one of SEQ ID NOs: 111, 123, 135, 147, 160, 173, 186, or 199.

11. The composition of claim 10, wherein the nucleic acid sequence comprises at least 90% sequence identity to any one of SEQ ID NOs: 11, 23, 35, 47, 60, 73, 86, or 99.

12. The composition of claim 1, wherein the DepD gene comprises a nucleic acid sequence encoding a polypeptide, wherein the polypeptide has at least 90% sequence identity to any one of SEQ ID NOs: 112, 124, 136, 148, 161, 174, 187, or 200.

13. The composition of claim 12, wherein the nucleic acid sequence comprises at least 90% sequence identity to any one of SEQ ID NOs: 12, 24, 36, 48, 61, 74, 87, or 100.

14. The composition of claim 1, wherein the DepE gene comprises a nucleic acid sequence encoding a polypeptide, wherein the polypeptide has at least 90% sequence identity to any one of SEQ ID NOs: 113, 125, 137, 149, 162, 175, 188, or 201.

15. The composition of claim 14, wherein the nucleic acid sequence comprises at least 90% sequence identity to any one of SEQ ID NOs: 13, 25, 37, 49, 62, 75, 88, or 101.

16. The composition of claim 1, wherein the DepF gene comprises a nucleic acid sequence encoding a polypeptide, wherein the polypeptide has at least 90% sequence identity to any one of SEQ ID NOs: 114, 126, 138, 150, 163, 176, 189, or 202.

17. The composition of claim 16, wherein the nucleic acid sequence comprises at least 90% sequence identity to any one of SEQ ID NOs: 14, 26, 38, 50, 63, 76, 89, or 102.

18. The composition of claim 1, wherein the DepG gene comprises a nucleic acid sequence encoding a polypeptide, wherein the polypeptide has at least 90% sequence identity to any one of SEQ ID NOs: 115, 127, 139, 151, 164, 177, 190, or 203.

19. The composition of claim 18, wherein the nucleic acid sequence comprises at least 90% sequence identity to any one of SEQ ID NOs: 15, 27, 39, 51, 64, 77, 90, or 103.

20. The composition of claim 1, wherein the DepH gene comprises a nucleic acid sequence encoding a polypeptide, wherein the polypeptide has at least 90% sequence identity to any one of SEQ ID NOs: 116, 128, 140, 152, 165, 178, 191, or 204.

21. The composition of claim 20, wherein the nucleic acid sequence comprises at least 90% sequence identity to any one of SEQ ID NOs: 16, 28, 40, 52, 65, 78, 91, or 104.

22. The composition of claim 1, wherein the DepI gene comprises a nucleic acid sequence encoding a polypeptide, wherein the polypeptide has at least 90% sequence identity to any one of SEQ ID NOs: 117, 129, 141, 153, 166, 179, 192, or 205.

23. The composition of claim 22, wherein the nucleic acid sequence comprises at least 90% sequence identity to any one of SEQ ID NOs: 17, 29, 41, 53, 66, 79, 92, or 105.

24. The composition of claim 1, wherein the DepJ gene comprises a nucleic acid sequence encoding a polypeptide, wherein the polypeptide has at least 90% sequence identity to any one of SEQ ID NOs: 118, 130, 142, 154, 167, 180, 193, or 206.

25. The composition of claim 24, wherein the nucleic acid sequence comprises at least 90% sequence identity to any one of SEQ ID NOs: 18, 30, 42, 54, 67, 80, 93, or 106.

26. The composition of claim 1, wherein the DepK gene comprises a nucleic acid sequence encoding a polypeptide, wherein the polypeptide has at least 90% sequence identity to any one of SEQ ID NOs: 143, 155, 168, 181, or 194.

27. The composition of claim 26, wherein the nucleic acid sequence comprises at least 90% sequence identity to any one of SEQ ID NOs: 43, 55, 68, 81, or 94.

28. The composition of claim 1, wherein the DepL gene comprises a nucleic acid sequence encoding a polypeptide, wherein the polypeptide has at least 90% sequence identity to any one of SEQ ID NOs: 119, 131, 156, 169, 182, 195, or 207.

29. The composition of claim 28, wherein the nucleic acid sequence comprises at least 90% sequence identity to any one of SEQ ID NOs: 19, 31, 43, 56, 69, 82, 95, or 107.

30. The composition of claim 1, wherein the DepM gene comprises a nucleic acid sequence encoding a polypeptide, wherein the polypeptide has at least 90% sequence identity to any one of SEQ ID NOs: 120, 132, 144, 157, 170, 183, 196, or 208.

31. The composition of claim 30, wherein the nucleic acid sequence comprises at least 90% sequence identity to any one of SEQ ID NOs: 20, 32, 44, 57, 70, 83, 96, or 108.

32. The composition of claim 1, wherein the composition further comprises a biocontrol agent selected from the group consisting of a bacteria, a fungus, a yeast, a protozoa, a virus, an entomopathogenic nematode, a botanical extract, a protein, a nucleic acid, a secondary metabolite, and an inoculant.

33. The composition of claim 1, wherein the composition further comprises an agrochemically active compound selected from the group consisting of an insecticide, a fungicide, a bactericide, and a nematicide.

34. The composition of claim 1, wherein the composition further comprises a compound selected from the group consisting of a safener, a lipo-chitooligosaccharide, a triglucosamine lipoglycine salt, an isoflavone, and a ryanodine receptor modulator.

35. The composition of claim 1, wherein the plant or the plant part is genetically modified.

36. A fermantate broth, wherein the fermentate broth is produced using a bacterial strain having a 16S rDNA sequence comprising at least 95% sequence identity to any one of SEQ ID NOs: 1-9, wherein the bacterial strain further comprises a DepA gene, a DepB gene, a DepC gene, a DepF gene, a DepG gene, DepH gene, a DepE gene, or a DepD gene, and wherein the fermentate broth has insecticidal activity.

37. The fermentate broth of claim 36, wherein the bacterial strain is a Pseudomonas chlororaphis, a Burkholderia rinojensis, or a Chromobacterium haemolyticum, wherein the Pseudomonas chlororaphis, Burkholderia rinojensis, or Chromobacterium haemolyticum.

38. The fermentate broth of claim 36, wherein the bacterial strain is a Pseudomonas chlororaphis strain SS532D1 (NRRL Deposit No. B-67638), a Pseudomonas chlororaphis strain SSP459B9-3 (NRRL Deposit No. B-67639), a Burkholderia rinojensis strain JH59178-1 (NRRL Deposit No. B-67640), a Chromobacterium haemolyticum strain JH91791-1 (NRRL Deposit No. B-67642), a Chromobacterium haemolyticum strain PMCJ4191H4-1 (NRRL Deposit No. B-67644), a Chromobacterium haemolyticum strain JH97285-1 (NRRL Deposit No. B-67641), or a Chromobacterium haemolyticum strain PMC3591F10-1 (NRRL Deposit No. B-67643), or a progeny, mutant, or variant thereof.

39. The fermentate broth of claim 36, wherein the fermentate broth comprises a depsipeptide produced by the bacterial strain as a metabolite.

40. The fermentate broth of claim 36, wherein the depsipeptide has the structure of FR901375, FK228, or a derivative or variant thereof.

41. The fermentate broth of claim 36, wherein the DepA gene comprises a nucleic acid sequence encoding a polypeptide, wherein the polypeptide has at least 90% sequence identity to any one of SEQ ID NOs: 109, 121, 133, 145, 158, 171, 184, or 197.

42. The fermentate broth of claim 41, wherein the nucleic acid sequence comprises at least 90% sequence identity to any one of SEQ ID NOs: 9, 21, 33, 45, 58, 71, 84, or 97.

43. The fermentate broth of claim 36, wherein the DepB gene comprises a nucleic acid sequence encoding a polypeptide, wherein the polypeptide has at least 90% sequence identity to any one of SEQ ID NOs: 110, 122, 134, 146, 159, 172, 185, or 198.

44. The fermentate broth of claim 43, wherein the nucleic acid sequence comprises at least 90% sequence identity to any one of SEQ ID NOs: 10, 22, 34, 46, 59, 72, 85, or 98.

45. The fermentate broth of claim 36, wherein the DepC gene comprises a nucleic acid sequence encoding a polypeptide, wherein the polypeptide has at least 90% sequence identity to any one of SEQ ID NOs: 111, 123, 135, 147, 160, 173, 186, or 199.

46. The fermentate broth of claim 45, wherein the nucleic acid sequence comprises at least 90% sequence identity to any one of SEQ ID NOs: 11, 23, 35, 47, 60, 73, 86, or 99.

47. The fermentate broth of claim 36, wherein the DepD gene comprises a nucleic acid sequence encoding a polypeptide, wherein the polypeptide has at least 90% sequence identity to any one of SEQ ID NOs: 112, 124, 136, 148, 161, 174, 187, or 200.

48. The fermentate broth of claim 47, wherein the nucleic acid sequence comprises at least 90% sequence identity to any one of SEQ ID NOs: 12, 24, 36, 48, 61, 74, 87, or 100.

49. The fermentate broth of claim 36, wherein the DepE gene comprises a nucleic acid sequence encoding a polypeptide, wherein the polypeptide has at least 90% sequence identity to any one of SEQ ID NOs: 113, 125, 137, 149, 162, 175, 188, or 201.

50. The fermentate broth of claim 49, wherein the nucleic acid sequence comprises at least 90% sequence identity to any one of SEQ ID NOs: 13, 25, 37, 49, 62, 75, 88, or 101.

51. The fermentate broth of claim 36, wherein the DepF gene comprises a nucleic acid sequence encoding a polypeptide, wherein the polypeptide has at least 90% sequence identity to any one of SEQ ID NOs: 114, 126, 138, 150, 163, 176, 189, or 202.

52. The fermentate broth of claim 51, wherein the nucleic acid sequence comprises at least 90% sequence identity to any one of SEQ ID NOs: 14, 26, 38, 50, 63, 76, 89, or 102.

53. The fermentate broth of claim 36, wherein the DepG gene comprises a nucleic acid sequence encoding a polypeptide, wherein the polypeptide has at least 90% sequence identity to any one of SEQ ID NOs: 115, 127, 139, 151, 164, 177, 190, or 203.

54. The fermentate broth of claim 53, wherein the nucleic acid sequence comprises at least 90% sequence identity to any one of SEQ ID NOs: 15, 27, 39, 51, 64, 77, 90, or 103.

55. The fermentate broth of claim 36, wherein the DepH gene comprises a nucleic acid sequence encoding a polypeptide, wherein the polypeptide has at least 90% sequence identity to any one of SEQ ID NOs: 116, 128, 140, 152, 165, 178, 191, or 204.

56. The fermentate broth of claim 55, wherein the nucleic acid sequence comprises at least 90% sequence identity to any one of SEQ ID NOs: 16, 28, 40, 52, 65, 78, 91, or 104.

57. The fermentate broth of claim 36, wherein the DepI gene comprises a nucleic acid sequence encoding a polypeptide, wherein the polypeptide has at least 90% sequence identity to any one of SEQ ID NOs: 117, 129, 141, 153, 166, 179, 192, or 205.

58. The fermentate broth of claim 57, wherein the nucleic acid sequence comprises at least 90% sequence identity to any one of SEQ ID NOs: 17, 29, 41, 53, 66, 79, 92, or 105.

59. The fermentate broth of claim 36, wherein the DepJ gene comprises a nucleic acid sequence encoding a polypeptide, wherein the polypeptide has at least 90% sequence identity to any one of SEQ ID NOs: 118, 130, 142, 154, 167, 180, 193, or 206.

60. The fermentate broth of claim 59, wherein the nucleic acid sequence comprises at least 90% sequence identity to any one of SEQ ID NOs: 18, 30, 42, 54, 67, 80, 93, or 106.

61. The fermentate broth of claim 36, wherein the DepK gene comprises a nucleic acid sequence encoding a polypeptide, wherein the polypeptide has at least 90% sequence identity to any one of SEQ ID NOs: 143, 155, 168, 181, or 194.

62. The fermentate broth of claim 61, wherein the nucleic acid sequence comprises at least 90% sequence identity to any one of SEQ ID NOs: 43, 55, 68, 81, or 94.

63. The fermentate broth of claim 36, wherein the DepL gene comprises a nucleic acid sequence encoding a polypeptide, wherein the polypeptide has at least 90% sequence identity to any one of SEQ ID NOs: 119, 131, 156, 169, 182, 195, or 207.

64. The fermentate broth of claim 63, wherein the nucleic acid sequence comprises at least 90% sequence identity to any one of SEQ ID NOs: 19, 31, 43, 56, 69, 82, 95, or 107.

65. The fermentate broth of claim 36, wherein the DepM gene comprises a nucleic acid sequence encoding a polypeptide, wherein the polypeptide has at least 90% sequence identity to any one of SEQ ID NOs: 120, 132, 144, 157, 170, 183, 196, or 208.

66. The fermentate broth of claim 65, wherein the nucleic acid sequence comprises at least 90% sequence identity to any one of SEQ ID NOs: 20, 32, 44, 57, 70, 83, 96, or 108.

67. A composition comprising the fermentate broth of claim 36, wherein the composition further comprises a biocontrol agent selected from the group consisting of a bacteria, a fungus, a yeast, a protozoa, a virus, an entomopathogenic nematode, a botanical extract, a protein, a nucleic acid, a secondary metabolite, and an innoculant.

68. A composition comprising the fermentate broth of claim 36, wherein the composition further comprises an agrochemically active compound selected from the group consisting of an insecticide, a fungicide, a bactericide, and a nematicide.

69. A composition comprising the fermentate broth of claim 36, wherein the composition further comprises a compound selected from the group consisting of a safener, a lipo-chitooligosaccharide, a triglucosamine lipoglycine salt, an isoflavone, and a ryanodine receptor modulator.

70. A composition comprising the fermentate broth of claim 36, wherein the plant or the plant part is genetically modified.

71. A composition comprising a depsipeptide and an agriculturally acceptable carrier, wherein the depsipeptide has insecticidal activity.

72. The composition of claim 71, wherein the depsipeptide has the structure of FR901375, FK228, or a derivative or variant thereof

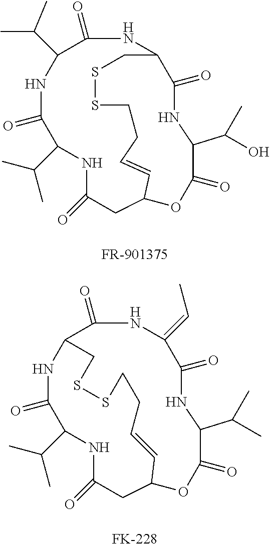

73. The composition of claim 71, wherein the composition further comprises a biocontrol agent selected from the group consisting of bacteria, a fungus, a yeast, protozoa, a virus, an entomopathogenic nematode, a botanical extract, a protein, a nucleic acid, a secondary metabolite, and an innoculant.

74. The composition of claim 71, wherein the composition further comprises an agrochemically active compound selected from the group consisting of an insecticide, a fungicide, a bactericide, and a nematicide.

75. The composition of claim 71, wherein the composition further comprises a compound selected from the group consisting of a safener, a lipo-chitooligosaccharide, a triglucosamine lipoglycine salt, an isoflavone, and a ryanodine receptor modulator.

76. The composition of claim 71, further comprising a plant or the plant part.

77. The composition of claim 76, wherein the plant or plant part comprises a seed.

78. The composition of claim 76, wherein the plant or the plant part is genetically modified.

79. A method for controlling a plant pathogen, pest, or insect comprising applying to a plant, a plant part, or an environment of a plant or a plant part bacterial strain or a fermentate broth produced by a bacterial strain, wherein the bacterial strain has a 16S rDNA sequence comprising at least 95% sequence identity to any one of SEQ ID NOs: 1-9, wherein the bacterial strain further comprises a DepA gene, a DepB gene, a DepC gene, a DepF gene, a DepG gene, DepH gene, a DepE gene, or a DepD gene, and wherein the bacterial strain has insecticidal activity.

80. The method of claim 79, wherein the bacterial strain is a Pseudomonas chlororaphis, a Burkholderia rinojensis, or a Chromobacterium haemolyticum, wherein the Pseudomonas chlororaphis, Burkholderia rinojensis, or Chromobacterium haemolyticum.

81. The method of claim 79, wherein the bacterial strain is a Pseudomonas chlororaphis strain SS532D1 (NRRL Deposit No. B-67638), a Pseudomonas chlororaphis strain SSP459B9-3 (NRRL Deposit No. B-67639), a Burkholderia rinojensis strain JH59178-1 (NRRL Deposit No. B-67640), a Chromobacterium haemolyticum strain JH91791-1 (NRRL Deposit No. B-67642), a Chromobacterium haemolyticum strain PMCJ4191H4-1 (NRRL Deposit No. B-67644), a Chromobacterium haemolyticum strain JH97285-1 (NRRL Deposit No. B-67641), or a Chromobacterium haemolyticum strain PMC3591F10-1 (NRRL Deposit No. B-67643), or a progeny, mutant, or variant thereof.

82. The method of claim 79, wherein the bacterial strain produces a depsipeptide as a metabolite.

83. The method of claim 79, wherein the depsipeptide has the structure of FR901375, FK228, or a derivative or variant thereof.

84. The method of claim 79, wherein the DepA gene comprises a nucleic acid sequence encoding a polypeptide, wherein the polypeptide has at least 90% sequence identity to any one of SEQ ID NOs: 109, 121, 133, 145, 158, 171, 184, or 197.

85. The method of claim 84, wherein the nucleic acid sequence comprises at least 90% sequence identity to any one of SEQ ID NOs: 9, 21, 33, 45, 58, 71, 84, or 97.

86. The method of claim 79, wherein the DepB gene comprises a nucleic acid sequence encoding a polypeptide, wherein the polypeptide has at least 90% sequence identity to any one of SEQ ID NOs: 110, 122, 134, 146, 159, 172, 185, or 198.

87. The method of claim 86, wherein the nucleic acid sequence comprises at least 90% sequence identity to any one of SEQ ID NOs: 10, 22, 34, 46, 59, 72, 85, or 98.

88. The method of claim 79, wherein the DepC gene comprises a nucleic acid sequence encoding a polypeptide, wherein the polypeptide has at least 90% sequence identity to any one of SEQ ID NOs: 111, 123, 135, 147, 160, 173, 186, or 199.

89. The method of claim 88, wherein the nucleic acid sequence comprises at least 90% sequence identity to any one of SEQ ID NOs: 11, 23, 35, 47, 60, 73, 86, or 99.

90. The method of claim 79, wherein the DepD gene comprises a nucleic acid sequence encoding a polypeptide, wherein the polypeptide has at least 90% sequence identity to any one of SEQ ID NOs: 112, 124, 136, 148, 161, 174, 187, or 200.

91. The method of claim 90, wherein the nucleic acid sequence comprises at least 90% sequence identity to any one of SEQ ID NOs: 12, 24, 36, 48, 61, 74, 87, or 100.

92. The method of claim 79, wherein the DepE gene comprises a nucleic acid sequence encoding a polypeptide, wherein the polypeptide has at least 90% sequence identity to any one of SEQ ID NOs: 113, 125, 137, 149, 162, 175, 188, or 201.

93. The method of claim 92, wherein the nucleic acid sequence comprises at least 90% sequence identity to any one of SEQ ID NOs: 13, 25, 37, 49, 62, 75, 88, or 101.

94. The method of claim 79, wherein the DepF gene comprises a nucleic acid sequence encoding a polypeptide, wherein the polypeptide has at least 90% sequence identity to any one of SEQ ID NOs: 114, 126, 138, 150, 163, 176, 189, or 202.

95. The method of claim 94, wherein the nucleic acid sequence comprises at least 90% sequence identity to any one of SEQ ID NOs: 14, 26, 38, 50, 63, 76, 89, or 102.

96. The method of claim 79, wherein the DepG gene comprises a nucleic acid sequence encoding a polypeptide, wherein the polypeptide has at least 90% sequence identity to any one of SEQ ID NOs: 115, 127, 139, 151, 164, 177, 190, or 203.

97. The method of claim 96, wherein the nucleic acid sequence comprises at least 90% sequence identity to any one of SEQ ID NOs: 15, 27, 39, 51, 64, 77, 90, or 103.

98. The method of claim 79, wherein the DepH gene comprises a nucleic acid sequence encoding a polypeptide, wherein the polypeptide has at least 90% sequence identity to any one of SEQ ID NOs: 116, 128, 140, 152, 165, 178, 191, or 204.

99. The method of claim 98, wherein the nucleic acid sequence comprises at least 90% sequence identity to any one of SEQ ID NOs: 16, 28, 40, 52, 65, 78, 91, or 104.

100. The method of claim 79, wherein the DepI gene comprises a nucleic acid sequence encoding a polypeptide, wherein the polypeptide has at least 90% sequence identity to any one of SEQ ID NOs: 117, 129, 141, 153, 166, 179, 192, or 205.

101. The method of claim 100, wherein the nucleic acid sequence comprises at least 90% sequence identity to any one of SEQ ID NOs: 17, 29, 41, 53, 66, 79, 92, or 105.

102. The method of claim 79, wherein the DepJ gene comprises a nucleic acid sequence encoding a polypeptide, wherein the polypeptide has at least 90% sequence identity to any one of SEQ ID NOs: 118, 130, 142, 154, 167, 180, 193, or 206.

103. The method of claim 102, wherein the nucleic acid sequence comprises at least 90% sequence identity to any one of SEQ ID NOs: 18, 30, 42, 54, 67, 80, 93, or 106.

104. The method of claim 79, wherein the DepK gene comprises a nucleic acid sequence encoding a polypeptide, wherein the polypeptide has at least 90% sequence identity to any one of SEQ ID NOs: 143, 155, 168, 181, or 194.

105. The method of claim 104, wherein the nucleic acid sequence comprises at least 90% sequence identity to any one of SEQ ID NOs: 43, 55, 68, 81, or 94.

106. The method of claim 79, wherein the DepL gene comprises a nucleic acid sequence encoding a polypeptide, wherein the polypeptide has at least 90% sequence identity to any one of SEQ ID NOs: 119, 131, 156, 169, 182, 195, or 207.

107. The method of claim 106, wherein the nucleic acid sequence comprises at least 90% sequence identity to any one of SEQ ID NOs: 19, 31, 43, 56, 69, 82, 95, or 107.

108. The method of claim 79, wherein the DepM gene comprises a nucleic acid sequence encoding a polypeptide, wherein the polypeptide has at least 90% sequence identity to any one of SEQ ID NOs: 120, 132, 144, 157, 170, 183, 196, or 208.

109. The method of claim 108, wherein the nucleic acid sequence comprises at least 90% sequence identity to any one of SEQ ID NOs: 20, 32, 44, 57, 70, 83, 96, or 108.

110. The method of claim 79, further comprising applying to the plant, plant part, seed, or environment of the plant or plant part with an agriculturally acceptable carrier.

111. The method of claim 79, wherein the composition further comprises applying to the plant, plant part, or environment of the plant or plant part a biocontrol agent selected from the group consisting of a bacteria, a fungus, a yeast, a protozoa, a virus, an entomopathogenic nematode, a botanical extract, a protein, a nucleic acid, a secondary metabolite, and an innoculant.

112. The method of claim 79, wherein the composition further comprises applying to the plant, plant part, or environment of the plant or the plant part an agrochemically active compound selected from the group consisting of an insecticide, a fungicide, a bactericide, and a nematicide.

113. The method of claim 79, wherein the composition further comprises applying to the plant, plant part, or environment of the plant or the plant part a compound selected from the group consisting of a safener, a lipo-chitooligosaccharide, a triglucosamine lipoglycine salt, an isoflavone, and a ryanodine receptor modulator.

114. The method of claim 79, wherein the plant, the plant part, or the environment of the plant or the plant part comprises a genetically modified plant, a genetically modified plant part, or an environment of a genetically modified plant or a genetically modified plant part.

115. A method for controlling a plant pathogen, pest, or insect comprising contacting a plant, a plant part, seed, or an environment of a plant, seed, or a plant part with a depsipeptide.

116. The method of claim 115, further comprising applying to the plant, plant part, or environment of the plant or plant part with an agriculturally acceptable carrier.

117. The method of claim 115, wherein the composition further comprises applying to the plant, plant part, or environment of the plant or plant part a biocontrol agent selected from the group consisting of bacteria, a fungus, a yeast, protozoa, a virus, an entomopathogenic nematode, a botanical extract, a protein, a nucleic acid, a secondary metabolite, and an innoculant.

118. The method of claim 115, wherein the composition further comprises applying to the plant, plant part, or environment of the plant or the plant part an agrochemically active compound selected from the group consisting of an insecticide, a fungicide, a bactericide, and a nematicide.

119. The method of claim 115, wherein the composition further comprises applying to the plant, plant part, or environment of the plant or the plant part a compound selected from the group consisting of a safener, a lipo-chitooligosaccharide, a triglucosamine lipoglycine salt, an isoflavone, and a ryanodine receptor modulator.

120. The method of claim 115, wherein the plant, the plant part, or the environment of the plant or the plant part comprises a genetically modified plant, a genetically modified plant part, or an environment of a genetically modified plant or a genetically modified plant part.

121. The method of claim 115, wherein the depsipeptide has the structure of FR901375, FK228, or a derivative or variant thereof.

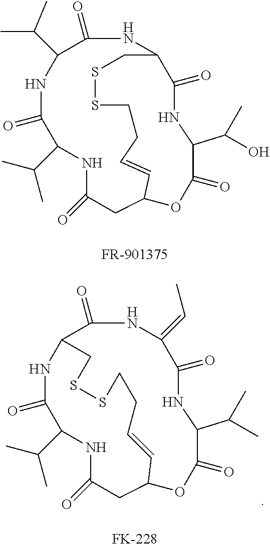

122.
